(19) United States
(12) Patent Application Publication
(10) Pub. No.: US 2024/0261304 A1
(43) Pub. Date: Aug. 8, 2024

(54) TERPENE-ENRICHED CANNABINOID COMPOSITION FOR TREATING CONDITIONS AND/ OR SYMPTOMS ASSOCIATED WITH A STRESSFUL EVENT

(71) Applicant: BUZZELET DEVELOPMENT AND TECHNOLOGIES LTD., OR AKIVA (IL)

(72) Inventors: Noa RAZ, Gizo (IL); Aharon M. EYAL, Jerusalem (IL); Ziv TURNER, Petach Tikva (IL)

(21) Appl. No.: 18/441,254

(22) Filed: Feb. 14, 2024

Related U.S. Application Data
(63) Continuation-in-part of application No. 17/068,845, filed on Oct. 13, 2020, now abandoned.
Application No. 17/068,845 is a continuation-in-part of application No. PCT/IB2019/053053, filed on Apr. 12, 2019.
(60) Provisional application No. 62/657,775, filed on Apr. 14, 2018.
(60) Provisional application No. 62/661,103, filed on Apr. 23, 2018.

(51) Int. Cl. A61K 31/00; A61K 45/06

(52) U.S. Cl. CPC A61K 31/658; A61K 45/06

(57) ABSTRACT

Preventing an occurrence of a condition or symptom thereof arising due to exposure to a stressful event, by administering to a subject in need thereof a therapeutically effective amount of a composition comprising at least one cannabinoid, at least one terpene selected from the group consisting of Pinene, Myrcene, Caryophyllene, Humulene, Nerolidol, Bisabolol, Linalool, Fenchol, Limonene, Terpinene, Terpineol, Eucalyptol, Geraniol, Borneol, Sabinene, Ocimene and combinations thereof, where the at least one terpene is present in the composition at a concentration of at least about 20 wt %, based on the weight of the total amount of terpenes in the composition and at least 5% by weight of a non-cannabinoid, non-terpene carrier.

### CROSS-REFERENCE TO RELATED APPLICATIONS

The present application claims the benefit of U.S. Provisional Application Ser. No. 62/657,775 filed Apr. 14, 2018 and U.S. Provisional Application Ser. No. 62/661,103 filed Apr. 23, 2018, the disclosures of which are expressly incorporated by reference herein in their entirety.

### FIELD OF THE INVENTION

The field of art to which this invention generally pertains is cannabinoid compositions, and specifically cannabinoid compositions for therapeutic use.

### BACKGROUND OF THE INVENTION

A stressful event represents a stress and/or distress event, situation, and/or gathering high in stressors, causing intrusion into ones' peaceful and/or routine or normal existence. A stressful event represents a situation causing an individual to feel worry, anxiety, tension. A stressful event may include pain and bodily injury. A stressful event is a distressing or disturbing experience, including surgery, dental treatment, unpleasant and/or painful medical procedure, sport injury, battlefield injury, a motor vehicle collision, gunshot, falls, sprain, joint dislocation, bone fracture, childbirth, stressful examination and/or task, stressful presentation, stressful social event, and combinations thereof. Physical pain and emotional stress may be immediate, characterizing the acute event, or prolonged or delayed, appearing after periods of time.

Conditions and/or symptoms that follow a stressful event may include stress, distress, anxiety (including acute or trauma-related anxiety), depression, acute stress, post traumatic stress, pain (including acute or trauma-related pain), neuronal injury, bacterial infection, muscle tension, inflammation and combination thereof. Such conditions may develop substantially immediately (i.e. within about 30 minutes of exposure to the stressful event) or even within up to about 6 months after such exposure.

A medical kit to be used in a stressful event should include medication and/or treatment composition to reduce anxiety and stress, and prevent and/or decrease post-trauma stress; to relief and/or prevent physical pain in cases of an injured or treated organ, including surgery, dentist treatment, unpleasant and/or painful medical procedure, sport injury, battlefield injury, a motor vehicle collision, gunshot, falls, sprain, joint dislocation, bone fracture, and/or delivery; to decrease muscle tension; to provide neuroprotection and prevent and/or reduce associated central and peripheral nervous system injury; to treat inflammation and possible bacterial infection at the injured or treated organ; and/or to increase the immune system response. Treatment is aimed to prevent, decrease and or treat stress, distress, anxiety, acute anxiety, depression, acute stress, post trauma stress, pain, acute pain, neuronal injury, bacterial infection, muscle tension, inflammation associated with the stressful event.

Terpenes play important roles in cannabinoids-comprising products, affecting the functionality and bioavailability of the cannabinoids and the aroma of the product. Processing cannabis plant material typically leads to terpenes loss so that most of cannabis products are of relatively low terpene content. That is particularly true for cannabis extracts and products thereof, such as cannabis tablets, cannabis gel capsules, cannabis patches, cannabis suppositories, etc. Most cannabis terpenes boiling points are in the range between about 150° C. and about 220° C. and they evaporate, at least partially, during cannabis buds drying, during solvent separation from extracts and during decarboxylation. Monoterpenes are lost at a rate greater than that of terpenes with a higher molecular weight and terpenes carrying no hydroxyl groups are lost at a rate greater than that of terpenes that do carry hydroxyl groups.

### SUMMARY OF THE INVENTION

According to an embodiment, provided is a terpene-enriched cannabinoid composition comprising (i) at least one cannabinoid in a specific amount, (ii) at least one primary terpene in a specific amount, wherein said at least one primary terpene forms at least 40% by weight of a total amount of terpenes in the composition, (iii) at least 5% by weight of a non-cannabinoid, non-terpene carrier, and (iv) optionally at least three secondary terpenes; and ((a) wherein the non-cannabinoid, non-terpene carrier comprises cellulose a weight/weight ratio of the total amount of terpenes to total cannabinoids in the composition is from about 0.1:1 to about 1:1, or (b) wherein the non-cannabinoid, non-terpene carrier comprises less than 5% by weight cellulose and a weight/weight ratio of the total amount of terpenes to total cannabinoids in the composition is about 0.05:1 to about 1:1.

According to an embodiment, provided is a terpene-enriched cannabinoid composition for treating conditions and/or symptoms associated with a stressful event comprising (i) at least one cannabinoid in a specific amount, (ii) at least one primary terpene in a specific amount, wherein said at least one primary terpene forms at least 40% by weight of a total amount of terpenes in the composition, (iii) at least 5% by weight of a non-cannabinoid, non-terpene carrier, and (iv) optionally at least three secondary terpenes; and ((a) wherein the non-cannabinoid, non-terpene carrier comprises cellulose a weight/weight ratio of the total amount of terpenes to total cannabinoids in the composition is from about 0.1:1 to about 1:1, or (b) wherein the non-cannabinoid, non-terpene carrier comprises less than 5% by weight cellulose and a weight/weight ratio of the total amount of terpenes to total cannabinoids in the composition is about 0.05:1 to about 1:1.

According to some embodiments, the composition has a therapeutic effect in treating conditions and/or symptoms associated with a stressful event, wherein the therapeutic effect is an enhanced therapeutic effect compared with that of a composition comprising the same cannabinoids amounts and one fifth the amount of the at least one primary terpene.

According to an embodiment, the composition comprises less than 5% by weight glycol.

According to an embodiment, the composition comprises a water concentration of less than 20% by weight.

According to an embodiment, the enhanced therapeutic effect is selected from the group consisting of a shortened onset time of the therapeutic effect, an increased magnitude of the therapeutic effect, an extended duration of the therapeutic effect, a reduced dosage of the compostion, a reduction in at least one secondary adverse symptom, a reduced frequency of the conditions and/or symptoms, a reduced severity of the conditions and/or symptoms, a reduced consumption of other drugs and combinations thereof.

According to an embodiment, the conditions and/or symptoms are selected from the group consisting of stress, distress, acute anxiety, trauma-related anxiety, depression, acute stress, post traumatic stress, acute pain, trauma-related pain, neuronal injury, bacterial infection, muscle tension, inflammation and combination thereof.

According to an embodiment, the therapeutic effect comprises an increased immune response.

According to an embodiment, the stressful event includes surgery, dental treatment, unpleasant and/or painful medical procedures, sports injuries, battlefield injuries, sprains, joint dislocations, bone fractures, childbirth, stressful examinations, stressful social events, trauma, major trauma, psychological trauma and combinations thereof.

According to an embodiment, the therapeutic effect comprises treatment of acute or sudden onset conditions and/or symptoms.

According to an embodiment, the therapeutic effect provides a first aid treatment.

According to an embodiment, the therapeutic effect treats delayed conditions and/or symptoms

According to an embodiment, the non-cannabinoid, non-terpene, carrier comprises cellulose, and the composition comprises (a) tetrahydrocannabinolic acid (THCa) in a concentration of at least 5% by weight; (b) cannabidiolic acid (CBDa) in a concentration of at least 5% by weight; or (c) tetrahydrocannabinolic acid (THCa) in a concentration of at least 2.5% by weight, and cannabidiolic acid (CBDa) in a concentration of at least 2.5% by weight; and (d) optionally at least one of cannabigerol (CBG), cannabinol (CBN), and cannabichromene (CBC) in a concentration of at least 0.5% by weight; and (i) the water concentration is about 20% to 5% by weight; (ii) the primary terpene forms at least 40% by weight of the total terpene content, and (iii) the primary terpene is selected from the group consisting of pinene, limonene, linalool, caryophyllene, caryophyllene oxide, myrcene, humulene, borneol, eucalyptol, terpineol, nerolidol, phytol, geraniol, bisabolol, camphene, beta-amyrin, thujone, citronellol, pulegone, cycloartenol, cymene, sabinene, carene, terpinene, fenchol, isopulegol, guaiol, phellandrene, eudesmol, ocimene, cardinene, elemene, gurjunene, farnesene and combinations thereof.

According to an embodiment, the composition comprises (a) at least one cannabinoid from the group consisting of THC, CBD, CBG, CBC, CBN and their non-decarboxylated form (b) less than 5% by weight cellulose; (c) less than 5% by weight water; (d) primary terpene forming at least 40% by weight of the total terpene content, and (e) primary terpene selected from the group consisting of pinene, limonene, linalool, caryophyllene, caryophyllene oxide, myrcene, humulene, borneol, eucalyptol, terpineol, nerolidol, phytol, geraniol, bisabolol, camphene, beta-amyrin, thujone, citronellol, pulegone, cycloartenol, cymene, sabinene, carene, terpinene, fenchol, isopulegol, guaiol, phellandrene, eudesmol, ocimene, cardinene, elemene, gurjunene, farnesene and combinations thereof.

According to an embodiment, the primary terpene is selected from the group consisting of pinene, limonene, linalool, caryophyllene, caryophyllene oxide, myrcene, humulene, borneol, eucalyptol, terpineol, nerolidol, phytol, geraniol, bisabolol, camphene, beta-amyrin, thujone, citronellol, pulegone, cycloartenol, cymene, sabinene, carene, terpinene, fenchol, isopulegol, guaiol, phellandrene, eudesmol, ocimene, cardinene, elemene, gurjunene, farnesene, friedelin, carvacrol, eugenol, camphor, menthol, iso-menthone, neral, gerial, viridiflorol, germacrene, thymol, Menth-2-en-1-ol, farensol, carotol, myrtenol and combinations thereof.

According to an embodiment, the shortened onset time of the therapeutic effect is a time at least 20% shorter than the onset time obtained with use of a composition comprising the same cannabinoids amounts and one fifth the amount of the at least one primary terpene.

According to an embodiment, the increased magnitude of the therapeutic effect is a magnitude of at least 20% greater than the magnitude obtained with a composition comprising the same cannabinoids amounts and one fifth the amount of the at least one primary terpene.

According to an embodiment, the extended duration of the therapeutic effect is a duration of at least 20% greater than the duration obtained with a composition comprising the same cannabinoids amounts and one fifth the amount of the at least one primary terpene.

According to an embodiment, a reduced requirement for the composition is a reduction of at least 20% in the dosage required to obtain the same therapeutic effect as that obtained with a dosage of a composition comprising the same cannabinoids amounts and one fifth the amount of the at least one primary terpene.

According to an embodiment, a reduction in at least one secondary adverse symptom is a decrease of at least 20% in a frequency, severity or extent of symptoms compared with a frequency, severity or extent of symptoms obtained with a composition comprising the same cannabinoids amounts and one fifth the amount of the at least one primary terpene.

According to an embodiment, a reduced severity of conditions and/or symptoms is a reduction of at least 20% compared with a severity of conditions and/or symptoms obtained with a composition comprising the same cannabinoids amounts and one fifth the amount of the at least one primary terpene.

According to an embodiment, a reduced consumption of other drugs is a reduction of at least 20% in the amount or frequency of other drugs consumed in order to obtain the same therapeutic effect compared with that obtained with that obtained by administering a composition comprising the same cannabinoids amounts and one fifth the amount of the at least one primary terpene.

According to an embodiment, reduced frequency of the conditions and/or symptoms is at least 20% smaller compared with that of a composition comprising the same cannabinoids amounts and one half the amount of the primary terpene.

According to an embodiment, reduced severity of the conditions and/or symptoms is a reduction of at least 20% compared with that of a composition comprising the same cannabinoids amounts and one fifth the amount of the at least one primary terpene.

According to an embodiment, the reduced consumption of other drugs is a reduction of at least 20% compared with that of a composition comprising the same cannabinoids amounts and one fifth the amount of the at least one primary terpene.

According to an embodiment, the at least one cannabinoid comprises at least one of CBD, THC, CBDa, THCa, CBG, CBC, CBN, CBL, THCV and CBDV and the at least one primary terpene is selected from the group consisting of linalool, limonene, eucalyptol, myrcene, caryophyllene, humulene, camphene, borneol, terpineol, pinene, terpinene, citronellol, amyrin, cycloartenol, camphor, sabinene, geraniol, guaiol, β-Eudesmol, menthol, bisabolol, fenchol, nerolidol, phellandrene, cymene, farnesene, citral and combinations thereof.

According to an embodiment, the at least one cannabinoid comprises at least one of CBD, THC, CBDa, THCa, CBG, CBC, CBN, CBN, CBL, THCV and CBDV, and the at least one primary terpene is selected from the group consisting of linalool, limonene, eucalyptol, caryophyllene, terpinene, humulene, terpineol, myrcene, nerolidol and combinations thereof.

According to an embodiment, the at least one cannabinoid comprises CBD and optionally THC at a CBD to THC weight/weight ratio of greater than 1:1, wherein the at least one primary terpene is selected from the group consisting of limonene, linalool, terpineol, citronellol, eucalyptol, caryophyllene, geraniol, menthol, pinene, bisabolol, borneol, fenchol, nerolidol and combinations thereof and the therapeutic effect comprises the treatment of anxiety and/or acute anxiety. According to some such embodiments, the at least one primary terpene does not comprises limonene or pinene and the composition comprises a secondary terpene selected from the group consisting of humulene, limonene, myrcene and pinene at a concentration of less than 5% by weight of the total terpene content.

According to an embodiment, the composition comprises CBD and optionally THC at a CBD to THC weight/weight ratio of greater than 1:1, wherein the at least one primary terpene is selected from the group consisting of linalool, limonene, humulene, terpineol, caryophyllene, bisabolol, borneol, fenchol and combination thereof, and the therapeutic effect comprises the treatment of acute stress and/or post traumatic stress. According to some such embodiments, the composition further comprises pinene and/or in an amount of less than 5% by weight of the total terpene content.

According to an embodiment, the composition comprises at least one of CBD, THC, CBC, CBN and CBG, the at least one primary terpene is selected from the group consisting of myrcene, linalool, humulene, cucalyptol, caryophyllene, caryophyllene oxide, amyrin, cycloartenol, borneol, sabinene, camphene, nerolidol, terpineol, geraniol, citronellol, camphor, bisabolol, limonene and combinations thereof and the therapeutic effect comprises the treatment of acute pain and/or trauma-related pain. According to some such embodiments, the at least one primary terpene is not selected from the group consisting of humulene, bisabolol, borneol, limonene and linalool, and the composition comprises a secondary terpene selected from the group consisting of humulene, bisabolol, borncol, limonene and linalool at a concentration of less than 5% weight of the total amont of terpenes.

According to an embodiment, the at least one cannabinoid is selected from the group consisitng of CBD, THC, THCa and CBG, and the at least one primary terpene is selected from the group consisting of caryophyllene, pinene, limonene, eucalyptol, myrcene, terpinene, humulene, citronellol, linalool, amyrin, cycloartenol, terpineol and combinations thereof and the therapeutic effect comprises neuroprotection.

According to an embodiment, the at least one cannabinoid is selected from the group consisting of CBD, THC, CBG, CBC and CBN, and the at least one primary terpene is selected from the group consisting of terpinolene, pinene, caryophyllene, eucalyptol, camphor, sabinene, humulene, phellandrene, cymene, linalool, amyrin, farnesene, borneol and combinations thereof and the therapeutic effect comprises antibacterial protection.

According to an embodiment, the at least one cannanbinoid is selected from the group consisting of CBD, THC, CBDa, THCa, CBC, CBG and CBN, and the at least one primary terpene is selected from the group consisting of myrcene, limonene, terpinene, pinene, eucalyptol, caryophyllene, guaiol, β-Eudesmol, humulene, citronellol, linalool, amyrin, cycolartenol and combinations thereof and the therapeutic effect comprises anti-inflammatory protection.

According to an embodiment, the composition comprises at least one of CBD, THC, CBDa, THCa, CBC, CBG and CBN, primary terpene is selected from the group consisting of terpinene, pinene, eucalyptol, caryophyllene and combinations thereof and the therapeutic effect increases anti-inflammatory protection.

According to an embodiment, the at least one cannabinoid is selected from the group consisting of CBD, THC and CBN, and the at least one primary terpene is selected from the group consisting of terpinolene, linalool, amyrin, pinene, borneol, caryophyllene, geraniol, limonene and combinations thereof and the enhanced therapeutic effect comprised increased immune response.

According to an embodiment, the at least one cannabinoid is selected from the group consistin of CBD, THC, THCa and CBN, and the at least one primary terpene is selected from the group consisting of myrcene, pinene, nerolidol, sabinene, linalool, humulene, caryophyllene, limonene, bisabolol, citral, eucalyptol, terpineol, geraniol and combinations thereof and the therapeutic effect comprises treatment of conditions and/or symptoms selected from the group consisting of muscle tension, cramps, and pain and combinatons thereof.

According to an embodiment, the at least one cannabinoid is selected from the group consistin of CBD, THC, and THCa, and the at least one primary terpene is selected from the group consisting of linalool, citronellol, geraniol, menthol, humulene, bisabolol, borneol, fenchol, linalool, limonene, terpineol, pinene, caryophyllene, myrcene, cymene, terpinene and combination thereof and the therapeutic effect comprises treatment of nausea and/or vomiting.

According to an embodiment, there is provided a preparation for treating symptoms associated with a stressful event comprising the composition as disclosed herein, wherein the composition is present at a concentration of between 1% by weight and 90% by weight of the total preparation.

According to an embodiment, the preparation further comprises an additive selected from the group consisting of antioxidants, emulsifiers and texturizers vegetable oils, plant extracts, honey, sucrose, glucose and fructose, pharmaceutical excipients and combinations thereof.

According to an embodiment, the preparation is provided in a form selected from the group consisting of tablets, gel capsules, medical patches, cigarettes, vaporizer liquids, topicals, creams, varnishes, sublingual oils, sprays, edibles, beverages, suppositories, rectal candles, nasal preparartions and combinations thereof.

According to an embodiment, the preparation is configured to provide the therapeutic effect with an onset time of less than 30 minutes.

According to an embodiment, there is provided a first aid kit comprising the preparation as disclosed herein.

According to an embodiment, the preparation is configured to provide the therapeutic effect with an onset time of more than 60 minutes.

According to an embodiment, there is provided a method for treating conditions and/or symptoms associated with a stressful event, comprising administering to a subject in need a therapeutically effective amount of a composition comprising (i) at least one cannabinoid in a specific amount, (ii) at least one primary terpene in a specific amount, wherein the at least one primary terpene is present at a concentration of at least 40% by weight of the total amount of terpenes in the composition, (iii) at least 5% by weight of a non-cannabinoid, non-terpene carrier, and (iv) optionally at least three secondary terpenes. According to an embodiment, the composition provides a therapeutic effect which is an enhanced therapeutic effect compared with that of a composition comprising the same cannabinoids amounts and one fifth the amount of the at least one primary terpene.

According to an embodiment, the composition comprises at least 5% by weight of a non-cannabinoid, non-terpene carrier. According to an embodiment, the composition comprises less than 5% by weight glycol. According to an embodiment, the composition comprises less than 20% by weight water. According to an embodiment, the non-cannabinoid, non-terpene carrier comprises cellulose and the terpenes to cannabinoids weight/weight ratio in the composition is about 0.1:1 to about 1:1, According to an embodiment, the non-cannabinoid, non-terpene carrier comprises less than 5% by weight cellulose and the terpenes to cannabinoids weight/weight ratio in the composition is about 0.05:1 to about 1:1.

According to an embodiment, the conditions and/or symptoms are selected from the group consisting of stress, distress, acute anxiety, trauma-related anxiety, depression, acute stress, post traumatic stress, acute pain, trauma-related pain, neuronal injury, bacterial infection, muscle tension, inflammation and combination thereof.

According to an embodiment, the enhanced therapeutic effect comprises an increased immune response.

According to an embodiment, the stressful event includes surgery, dental treatment, unpleasant and/or painful medical procedure, sport injury, battlefield injury, traumatic injury, sudden physical injury, sprain, joint dislocation, bone fracture, childbirth, stressful examination, stressful social event, trauma, major trauma, psychological trauma and combination thereof.

According to an embodiment, the therapeutic effect comprises treatment of acute or sudden onset conditions and/or symptoms.

According to an embodiment, the therapeutic effect comprises a first aid treatment.

According to an embodiment, the conditions and/or symptoms comprise delayed conditions and/or symptoms

According to an embodiment, the composition further comprises at least one additive selected from the group consisting of antioxidants, emulsifiers and texturizers vegetable oils, plant extracts, honey, sucrose, glucose and fructose, pharmaceutical excipients and combinations thereof.

According to an embodiment, the non-cannabinoid, non-terpene, carrier comprises cellulose, and the composition comprises (a) tetrahydrocannabinolic acid (THCa) in a concentration of at least 5% by weight; (b) cannabidiolic acid (CBDa) in a concentration of at least 5% by weight; or (c) tetrahydrocannabinolic acid (THCa) in a concentration of at least 2.5% by weight, and cannabidiolic acid (CBDa) in a concentration of at least 2.5% by weight, and (d) optionally at least one of cannabigerol (CBG), cannabinol (CBN), and cannabichromene (CBC) in a concentration of at least 0.5% by weight (i); and the water concentration is about 20% to 5% by weight; (ii) the primary terpene forms at least 40% by weight of the total terpene content, and (iii) the primary terpene is selected from the group consisting of pinene, limonene, linalool, caryophyllene, caryophyllene oxide, myrcene, humulene, borneol, eucalyptol, terpineol, nerolidol, phytol, geraniol, bisabolol, camphene, beta-amyrin, thujone, citronellol, pulegone, cycloartenol, cymene, sabinene, carene, terpinene, fenchol, isopulegol, guaiol, phellandrene, eudesmol, ocimene, cardinene, elemene, gurjunene, farnesene and combinations thereof.

According to an embodiment, the composition comprises (a) at least one cannabinoid from the group consisting of THC, CBD, CBG, CBC, CBN and their non-decarboxylated form (b) less than 5% by weight cellulose; (c) less than 5% by weight water; (d) primary terpene forming at least 40% by weight of the total terpene content, and (e) primary terpene selected from the group consisting of pinene, limonene, linalool, caryophyllene, caryophyllene oxide, myrcene, humulene, borneol, eucalyptol, terpineol, nerolidol, phytol, geraniol, bisabolol, camphene, beta-amyrin, thujone, citronellol, pulegone, cycloartenol, cymene, sabinene, carene, terpinene, fenchol, isopulegol, guaiol, phellandrene, eudesmol, ocimene, cardinene, elemene, gurjunene, farnesene and combinations thereof.

According to an embodiment, the primary terpene is selected from the group consisting of pinene, limonene, linalool, caryophyllene, caryophyllene oxide, myrcene, humulene, borneol, eucalyptol, terpineol, nerolidol, phytol, geraniol, bisabolol, camphene, beta-amyrin, thujone, citronellol, pulegone, cycloartenol, cymene, sabinene, carene, terpinene, fenchol, isopulegol, guaiol, phellandrene, eudesmol, ocimene, cardinene, elemene, gurjunene, farnesene, friedelin, carvacrol, eugenol, camphor, menthol, iso-menthone, neral, gerial, viridiflorol, germacrene, thymol, Menth-2-en-1-ol, farensol, carotol, myrtenol and combinations thereof.

According to an embodiment, the enhanced therapeutic effect is selected from the group consisting of a shortened onset time of said therapeutic effect, an increased magnitude of said therapeutic effect, an extended duration of said therapeutic effect, a reduced requirement for said composition, a reduction in at least one secondary adverse symptom, a reduced frequency of said conditions and/or symptoms, a reduced severity of said conditions and/or symptoms, a reduced requirement for other drugs and combinations thereof.

According to an embodiment, the shortened onset time of the therapeutic effect is a time at least 20% shorter than the onset time obtained by administering a composition comprising the same cannabinoids amounts and one fifth the amount of the at least one primary terpene.

According to an embodiment, the increased magnitude of the therapeutic effect is a magnitude of at least 20% greater than the magniture obtained by administering a composition comprising the same cannabinoids amounts and one fifth the amount of the at least one primary terpene.

According to an embodiment, the extended duration of the therapeutic effect is a duration of at least 20% greater than the duration obtained by administering a composition comprising the same cannabinoids amounts and one fifth the amount of the at least one primary terpene.

According to an embodiment, the reduced requirement for the composition is a reduction of at least 20% in the dosage required to obtain the same therapeutic effect as that obtained by administering a dosage of a composition comprising the same cannabinoids amounts and one fifth the amount of the at least one primary terpene.

According to an embodiment, a reduction in at least one secondary adverse symptom is a decrease of at least 20% in a frequency, severity or extent of the adverse symptoms compared with a frequency, severity or extent of the adverse symptoms obtained with a composition comprising the same cannabinoids amounts and one fifth the amount of the at least one primary terpene.

Acording to an embodiment, the reduced severity of the conditions and/or symptoms is a reduction of at least 20% compared with the severity of the conditions and/or symptoms obtained by administering a composition comprising the same cannabinoids amounts and one fifth the amount of the at least one primary terpene.

According to an embodiment, the reduced requirement for other drugs is a reduction of at least 20% in the amount or frequency of other drugs required to obtain the same therapeutic effect compared with that obtained by administering a composition comprising the same cannabinoids amounts and one fifth the amount of the at least one primary terpene.

According to an embodiment, the frequency of the conditions and/or symptoms is a reduction of at least 20% compared with the frequency of the conditions and/or symptoms obtained by administering a composition comprising the same cannabinoids amounts and one fifth the amount of the at least one primary terpene.

According to an embodiment, the reduced severity of the conditions and/or symptoms is a reduction of at least 20% compared with that of administering a composition comprising the same cannabinoids amounts and one fifth the amount of the at least one primary terpene.

According to an embodiment, the decreased consumption of other drugs is a reduction of at least 20% in the amount or frequency of other drugs consumed in order to obtain the same therapeutic effect compared with that obtained by administering a composition comprising the same cannabinoids amounts and one half the amount of the at least one primary terpene.

According to an embodiment, at least one cannanbinoid is selected from the group consisting of CBD, THC, CBDa, THCa, CBG, CBC and CBN, wherein the at least one primary terpene is selected from the group consisting of linalool, limonene, eucalyptol, myrcene, caryophyllene, humulene, camphene, borneol, terpineol, pinene, terpinene, citronellol, amyrin, cycloartenol, camphor, sabinene, geraniol, guaiol, β-Eudesmol, menthol, bisabolol, fenchol, nerolidol, phellandrene, cymene, farnesene, citraland combinations thereof.

According to an embodiment, the at least one cannabinoid is selected from the group consisting of CBD, THC, CBDa, THCa, CBG, CBC and CBN, wherein the at least one primary terpene is selected from the group consisting of linalool, limonene, eucalyptol, caryophyllene, terpinene, humulene, terpineol, myrcene, nerolidol and combinations thereof.

According to an embodiment, the at least one cannabinoid is selected from the group consisting of CBD and optionally, THC, at a CBD to THC weight/weight ratio of greater than 1:1, wherein the at least one primary terpene is selected from the group consisting of limonene, linalool, terpineol, citronellol, eucalyptol, caryophyllene, geraniol, menthol, pinene, bisabolol, borneol, fenchol, nerolidol and combinations thereof and the therapeutic effect comprises the treatment of acute anxiety and/or trauma-related anxiety.

According to one such embodiment, the at least one primary terpene does not comprise limone or pinene, and the composition comprises a secondary terpene selected from the group consisting of humulene, limonene, myrcene and pinene at a concentration of less than 5% by weight of the total terpene content.

According to an embodiment, the at least one cannabinoid is selected from the group consisting of CBD, and optionally THC at a CBD to THC weight/weight ratio of greater than 1:1, wherein said at least one primary terpene is selected from the group consisting of linalool, limonene, humulene, terpineol, caryophyllene, bisabolol, borneol, fenchol and combination thereof, and the therapeutic effect comprises the treatment of acute stress and/or post traumatic stress.

According to an embodiment, the composition further comprises pinene and/or eucalyptol at a concentration of less than 5% by weight of the total terpene content.

According to an embodiment, the at least one cannabinoid is selected from the group consisting of CBD, THC, CBC, CBN and CBG, wherein the at least one primary terpene is selected from the group consisting of myrcene, linalool, humulene, eucalyptol, caryophyllene, caryophyllene oxide, amyrin, cycloartenol, borneol, sabinene, camphene, nerolidol, terpineol, geraniol, citronellol, camphor, bisabolol, limonene and combinations thereof and the therapeutic effect comprises the treatment of acute pain and/or trauma-related pain.

According to some such embodiments, the at least one primary terpene is not selected from the group consisting of humulene, bisabolol, borneol, limonene and linalool, and the composition comprises a secondary terpene selected from the group consisting of humulene, bisabolol, borneol, limonene and linalool at a concentration of less than 5% by weight of the total amount of terpenes.

According to an embodiment, the at least one cannabinoid is selected from the group consisting of CBD, THC, THCa and CBG, wherein the at least one primary terpene is selected from the group consisting of caryophyllene, pinene, limonene, eucalyptol, myrcene, terpinene, humulene, citronellol, linalool, amyrin, cycloartenol, terpineol and combinations thereof and the therapeutic effect comprises neuroprotection.

According to an embodiment, the at least one cannabinoid is selected from the group consisting of CBD, THC, CBG, CBC and CBN, wherein the at least one primary terpene is selected from the group consisting of terpinolene, pinene, caryophyllene, eucalyptol, camphor, sabinene, humulene, phellandrene, cymene, linalool, amyrin, farnesene, borneol and combinations thereof and the therapeutic effect comprises antibacterial protection.

According to an embodiment, the s at least one cannabinoid is selected from the group consisting of CBD, THC, CBDa, THCa, CBC, CBG and CBN, wherein the at least one primary terpene is selected from the group consisting of myrcene, limonene, terpinene, pinene, eucalyptol, caryophyllene, guaiol, β-Eudesmol, humulene, citronellol, linalool, amyrin, cycolartenol and combinations thereof and the therapeutic effect comprises anti-inflammatory protection.

According to an embodiment, the at least one cannabinoid is selected from the group consisting of CBD, THC and CBN, wherein the at least one primary terpene is selected from the group consisting of terpinolene, linalool, amyrin, pinene, borneol, caryophyllene, geraniol, limonene and combinations thereof and the enhanced therapeutic effect comprises increased immune response.

According to an embodiment, the at least one cannabinoid is selected from the group consisting of CBD, THC, THCa and CBN, wherein the at least one primary terpene is selected from the group consisting of myrcene, pinene, nerolidol, sabinene, linalool, humulene, caryophyllene, limonene, bisabolol, citral, eucalyptol, terpineol, geraniol and combinations thereof and the therapeutic effect comprises treatment of conditions and/or symptoms selected from the group consisting of muscle tension, cramps, and pain and combinations thereof.

According to an embodiment, the at least one cannabinoid is selected from the group consisting of CBD, THC, and THCa, wherein the at least one primary terpene is selected from the group consisting of linalool, citronellol, geraniol, menthol, humulene, bisabolol, borneol, fenchol, linalool, limonene, terpineol, pinene, caryophyllene, myrcene, cymene, terpinene and combination thereof and the therapeutic effect comprises treatment of nausea and/or vomiting.

According to an embodiment, the onset time of the therapeutic effect is less than 30 minutes.

According to an embodiment, the onset time of the therapeutic effect is greater than 60 minutes.

According to an embodiment, the method comprises administering to the subject for a first period of time a first composition comprising a first cannabinoid at a first cannabinoid amount and at least one first primary terpene at a first primary terpene amount, followed by at least one selected from the group consisting of:
administering to said subject for a second period of time a second said composition comprising said first cannabinoid at said first cannabinoid amount or less and a second primary terpene at a second primary terpene amount;
administering to said subject for a second period of time a second said compositon comprising said first cannabinoid at said first cannabinoid amount or less and said first primary terpene at an increased first primary terpene amount;
administering to said subject for a second period of time a second said composition comprising said first cannabinoid at said first cannabinoid amount or less, said first primary terpene at said first primary terpene amount and at least one secondary terpene; and
administering to said subject for a second period of time a second said composition comprising said first cannabinoid at said first cannabinoid amount or less, said first primary terpene at said first primary terpene amount and a second primary terpene.

According to an embodiment, further provided is a method for treating conditions and/or symptoms associated with a stressful event, comprising administering to a subject in need a therapeutically effective amount of a composition comprising (i) at least one primary terpene in a specific amount; (ii) at least 5% by weight of a non-cannabinoid, non-terpene carrier; (iii) optionally at least three secondary terpenes; and (iv) optionally at least one cannabinoid in a specific amount,

According to an embodiment, the composition provides a therapeutic effect which is an enhanced therapeutic effect compared with that of a composition comprising the same cannabinoids amounts and one fifth the amount of said the at least one primary terpene.

According to an embodiment, the composition comprises less than 20% by weight water,

According to an embodiment, the conditions and/or symptoms are selected from the group consisting of stress, distress, acute anxiety, trauma-related anxiety, depression, acute stress, post traumatic stress, acute pain, trauma-related pain, neuronal injury, bacterial infection, muscle tension, inflammation and combination thereof.

According to an embodiment, the enhanced therapeutic effect increases comprises an increased immune response.

According to an embodiment, the stressful event includes surgery, dental treatment, unpleasant and/or painful medical procedure, sport injury, battlefield injury, traumatic injury, sudden physical injury, sprain, joint dislocation, bone fracture, childbirth, stressful examination, stressful social event, trauma, psychological trauma and combination thereof.

According to an embodiment, the therapeutic effect treats acute or sudden onset conditions and/or symptoms.

According to an embodiment, the conditions and/or symptoms comprise delayed conditions and/or symptoms.

According to an embodiment, the composition further comprises an additive selected from the group consisting of antioxidants, emulsifiers and texturizers vegetable oils, plant extracts, honey, sucrose, glucose and fructose, pharmaceutical excipients and combinations thereof.

According to an embodiment, the primary terpene is selected from the group consisting of pinene, limonene, linalool, caryophyllene, caryophyllene oxide, myrcene, humulene, borneol, eucalyptol, terpineol, nerolidol, phytol, geraniol, bisabolol, camphene, beta-amyrin, thujone, citronellol, pulegone, cycloartenol, cymene, sabinene, carene, terpinene, fenchol, isopulegol, guaiol, phellandrene, eudesmol, ocimene, cardinene, elemene, gurjunene, farnesene, friedelin, carvacrol, eugenol, camphor, menthol, iso-menthone, neral, gerial, viridiflorol, germacrene, thymol, Menth-2-en-1-ol, farensol, carotol, myrtenol and combinations thereof.

According to an embodiment, the enhanced therapeutic effect is selected from the group consisting of a shortened onset time of said therapeutic effect, an increased magnitude of said therapeutic effect, an extended duration of said therapeutic effect, a reduced requirement for said composition, a reduction in at least one secondary adverse symptom, a reduced frequency of said conditions and/or symptoms, a reduced severity of said conditions and/or symptoms, a reduced requirement for other drugs and combinations thereof.

According to an embodiment, the shortened onset time of the therapeutic effect is a time at least 20% shorter than the onset time obtained by administering a composition comprising the same cannabinoids amounts and one fifth the amount of the at least one primary terpene.

According to an embodiment, the increased magnitude of the therapeutic effect is a magnitude of at least 20% greater than the magniture obtained by administering a composition comprising the same cannabinoids amounts and one fifth the amount of the at least one primary terpene.

According to an embodiment, the extended duration of the therapeutic effect is a duration of at least 20% greater than the duration obtained by administering a composition comprising the same cannabinoids amounts and one fifth the amount of the at least one primary terpene.

According to an embodiment, the reduced requirement for the composition is a reduction of at least 20% in the dosage required to obtain the same therapeutic effect as that obtained by administering a dosage of a composition comprising the same cannabinoids amounts and one fifth the amount of the at least one primary terpene.

According to an embodiment, a reduction in at least one secondary adverse symptom is a decrease of at least 20% in a frequency, severity or extent of the adverse symptoms compared with a frequency, severity or extent of the adverse symptoms obtained with a composition comprising the same cannabinoids amounts and one fifth the amount of the at least one primary terpene.

Acording to an embodiment, the reduced severity of the conditions and/or symptoms is a reduction of at least 20% compared with the severity of the conditions and/or symptoms obtained by administering a composition comprising the same cannabinoids amounts and one fifth the amount of the at least one primary terpene.

According to an embodiment, the reduced requirement for other drugs is a reduction of at least 20% in the amount or frequency of other drugs required to obtain the same therapeutic effect compared with that obtained by administering a composition comprising the same cannabinoids amounts and one fifth the amount of the at least one primary terpene.

According to an embodiment, the frequency of the conditions and/or symptoms is a reduction of at least 20% compared with the frequency of the conditions and/or symptoms obtained by administering a composition comprising the same cannabinoids amounts and one fifth the amount of the at least one primary terpene.

According to an embodiment, the reduced severity of the conditions and/or symptoms is a reduction of at least 20% compared with that of administering a composition comprising the same cannabinoids amounts and one fifth the amount of the at least one primary terpene.

According to an embodiment, the decreased consumption of other drugs is a reduction of at least 20% in the amount or frequency of other drugs consumed in order to obtain the same therapeutic effect compared with that obtained by administering a composition comprising the same cannabinoids amounts and one half the amount of the at least one primary terpene.

According to an embodiment, the primary terpene is selected from the group consisting of at least two, at least three, at least four or at least five of linalool, limonene, eucalyptol, myrcene, caryophyllene, humulene, camphene, borneol, terpineol, pinene, terpinene, citronellol, amyrin, cycloartenol, camphor, sabinene, geraniol, guaiol, β-Eudesmol, menthol, bisabolol, fenchol, nerolidol, phellandrene, cymene, farnesene, citral and combinations thereof.

According to an embodiment, the primary terpene is selected from the group consisting of at least two, at least three, at least four, at least five, at least six, at least seven, at least eight or all nine of linalool, limonene, eucalyptol, caryophyllene, terpinene, humulene, terpineol, myrcene, nerolidol and combinations thereof.

According to an embodiment, the at least one primary terpene is selected from the group consisting of two or three of limonene, linalool, terpineol, citronellol, eucalyptol, caryophyllene, geraniol, menthol, pinene, bisabolol, borneol, fenchol, nerolidol, and the therapeutic effect comprises treatment of acute anxiety and/or trauma-related anxiety.

According to some such embodiments, at least one primary terpene does not comprise limonene or pinene, and the composition comprises a secondary terpene selected from the group consisting of humulene, limonene, myrcene and pinene at a concentration of less than 5% by weight of said total amount of terpenes.

According to an embodiment, the at least one primary terpene is selected from the group consisting of linalool, limonene, humulene, terpineol, caryophyllene, bisabolol, borneol, fenchol and combination thereof, and the therapeutic effect treats acute stress and/or post traumatic stress.

According to some such embodiments, the composition further comprises pinene and/or eucalyptol at a concentration of less than 5% by weight of the total terpene content.

According to an embodiment, the at least one primary terpene is selected from the group consisting of at least two, at least three, at least four or at least five of myrcene, linalool, humulene, eucalyptol, caryophyllene, caryophyllene oxide, amyrin, cycloartenol, borneol, sabinene, camphene, nerolidol, terpineol, geraniol, citronellol, camphor, bisabolol, limonene, and wherein the conditions and/or symptoms comprise acute pain and/or trauma-related pain.

According to some such embodiments, the at least one primary terpene is not selected from the group consisting humulene, bisabolol, borneol, limonene and linalool, and the composition comprises a secondary terpene selected from the group consisting of humulene, bisabolol, borneol, limonene and linalool at a concentration of less than 5% by weight of said total amount of terpenes.

According to an embodiment, the at least one primary terpene is selected from the group consisting of at least two, at least three, at least four or at least five of caryophyllene, pinene, limonene, eucalyptol, myrcene, terpinene, humulene, citronellol, linalool, amyrin, cycloartenol, terpeniol and the therapeutic effect comprises neuroprotection.

According to an embodiment, the at least one primary terpene is selected from the group consisting of at least two, at least three, at least four or at least five of terpinolene, pinene, caryophyllene, eucalyptol, camphor, sabinene, humulene, phellandrene, cymene, linalool, amyrin, farnesene, borneol and the therapeutic effect comprises antibacterial protection.

According to an embodiment, the at least one primary terpene is selected from the group consisting of at least two, at least three, at least four or at least five of myrcene, limonene, terpinene, pinene, eucalyptol, caryophyllene, guaiol, β-Eudesmol, humulene, citronellol, linalool, amyrin, cycolartenol and the therapeutic effect comprises anti-inflammatory protection.

According to an embodiment, the at least one primary terpene is selected from the group consisting of at least two, at least three, at least four or at least five of terpinolene, linalool, amyrin, pinene, borneol, caryophyllene, geraniol and the enhanced therapeutic effect comprises increased immune response.

According to an embodiment, the at least one primary terpene is selected from the group consisting of at least two, at least three, at least four, at least five, at least six, at least seven, at least eight, at least nine, at least ten, at least eleven, at least twelve, or all thirteen of myrcene, pinene, nerolidol, sabinene, linalool, humulene, caryophyllene, limonene, bisabolol, citral, eucalyptol, terpineol, geraniol and the conditions and/or symptoms are selected from the group consisting of muscle tension, cramps, and pain or combinations thereof.

According to an embodiment, the at least one primary terpene is selected from the group consisting of at least two, at least three, at least four, at least five, at least six, at least seven, at least eight, at least nine, at least ten, at least eleven, at least twelve, at least thirteen, at least fourteen, at least fifteen, or all sixteen of linalool, citronellol, geraniol, menthol, humulene, bisabolol, borneol, fenchol, linalool, limonene, terpineol, pinene, caryophyllene, myrcene, cymene, terpinene and the conditions and/or symptoms comprise nausea and/or vomiting.

According to an embodiment, the therapeutic effect has an onset time of less than 30 minutes.

According to an embodiment, the therapeutic effect has an onset time of greater than 60 minutes.

According to an aspect of some embodiments of the present invention, there is provided a method for preventing the occurrence of conditions or symptoms thereof arising due to exposure to a stressful event, comprising administering to a subject in need thereof a therapeutically effective amount of a composition comprising at least one cannabinoid; at least one terpene selected from the group consisting of Pinene, Myrcene, Caryophyllene, Humulene, Nerolidol, Bisabolol, Linalool, Fenchol, Limonene, Terpinene, Terpineol, Eucalyptol, Geraniol, Borneol, Sabinene, Ocimene and combinations thereof, wherein said at least one terpene is present in the composition at a concentration of at least about 20 wt %, based on the weight of the total amount of terpenes in the composition. and at least 5% by weight of a non-cannabinoid, non-terpene carrier.

According to an embodiment, a weight/weight ratio of a total amount of terpenes/total amount of cannabinoids in said composition is from 0.05 to 1.

According to an embodiment, the composition is administered prior to exposure of the subject to the stressful event, such as at least 5 minutes, 10 minutes, 15 minutes, 30 minutes, 1 hour, 2 hours, 3 hours, 4 hours, 5 hours, 6 hours, 7 hours, 8 hours, 9 hours or 10 hours prior to exposure to the stressful event.

According to an embodiment, the composition is administered subsequent to exposure to the stressful event and prior to occurrence of the conditions and symptoms. According to some embodiments, the composition is administered within 5 minutes, 10 minutes, 15 minutes, 30 minutes, 1 hour, 2 hours, 3 hours, 4 hours, 5 hours, 6 hours, 7 hours, 8 hours, 9 hours or 10 hours subsequent to exposure to the stressful event, but prior to the occurence of any detectable conditions or symptoms.

According to an embodiment, the composition has an onset time of less than about 30 minutes, such as less than about 30, less than about 25, less than about 20, less than about 15 or less than about 10 minutes.

According to an embodiment, the composition has an onset time of greater than about 60 minutes, such as greater than about 60 minutes, greater than about 1 hour, 2 hours, 3 hours, 4 hours, 5 hours, 6 hours, 7 hours, 8 hours, 9 hours or 10 hours.

According to an embodiment, said composition is liquid at 30° C.

According to an embodiment, said at least one cannabinoid is selected from the group consisting of CBD, THC, CBDa, THCa, CBG, CBC, CBN, CBL, THCV, CBDV and combinations thereof.

According to an embodiment, said composition provides a therapeutic effect which is an enhanced therapeutic effect compared with that of a composition comprising one half the amount of said at least one terpene.

According to an embodiment, said conditions or symptoms thereof are selected from the group consisting of acute anxiety, trauma-related anxiety, depression, acute stress, post traumatic stress, acute pain, trauma-related pain, neuronal injury, bacterial infection, muscle tension, inflammation, poor immune response, nausea and/or vomiting and combinations thereof.

According to an embodiment, the stressful event is selected from the group consisting of surgery, dental treatment, unpleasant and/or painful medical procedure, sports injury, battlefield injury, traumatic injury, sudden physical injury, automobile accident, work-related accident, sprain, joint dislocation, bone fracture, childbirth, stressful examination, stressful social event, trauma, psychological trauma and combinations thereof.

According to an embodiment, said conditions or symptoms thereof are selected from the group consisting of acute onset conditions or symptoms thereof; sudden onset conditions or symptoms thereof; and delayed onset conditions or symptoms thereof.

According to an embodiment, said at least one cannabinoid comprises CBD and optionally THC at a CBD to THC weight/weight ratio of greater than 1:1, wherein said at least one terpene is selected from the group consisting of limonene, linalool, terpineol, eucalyptol, caryophyllene, geraniol, pinene, bisabolol, fenchol, nerolidol and combinations thereof and wherein said condition or symptom thereof is selected from the group consisting of depression, acute anxiety, trauma-related anxiety and combinations thereof.

According to an embodiment, said at least one cannabinoid comprises CBD and optionally THC at a CBD to THC weight/weight ratio of greater than 1:1, wherein said at least one terpene is selected from the group consisting of linalool, limonene, humulene, terpineol, caryophyllene, bisabolol, fenchol and combination thereof and wherein said condition or symptom thereof comprises acute stress and/or post traumatic stress.

According to an embodiment, said at least one cannabinoid is selected from the group consisting of CBD, THC, CBC, CBN and CBG, wherein said at least one terpene is selected from the group consisting of myrcene, linalool, humulene, eucalyptol, caryophyllene, nerolidol, terpineol, geraniol, bisabolol, limonene, borneol, sabinene, ocimene and combinations thereof and wherein said condition or symptom thereof comprises acute pain and/or trauma-related pain.

According to an embodiment, said at least one cannabinoid is selected from the group consisting of CBD, THC, THCa and CBG, wherein said at least one terpene is selected from the group consisting of caryophyllene, pinene, limonene, eucalyptol, myrcene, terpinene, humulene, linalool, terpineol, borneol, sabinene, ocimene and combinations thereof and wherein said condition or symptom thereof comprises neuronal injury.

According to an embodiment, said at least one cannabinoid is selected from the group consisting of CBD, THC, CBG, CBC and CBN, wherein said at least one terpene is selected from the group consisting of terpinene, pinene, caryophyllene, eucalyptol, humulene, linalool, limonene, bisabolol and combinations thereof and wherein said condition or symptom therof comprises bacterial infection.

According to an embodiment, said at least one cannabinoid is selected from the group consisting of CBD, THC, CBDa, THCa, CBC, CBG and CBN, wherein said at least one terpene is selected from the group consisting of myrcene, limonene, terpinene, pinene, eucalyptol, caryophyllene, humulene, linalool, bisabolol and combinations thereof and wherein said condition or symptom thereof comprises inflammation.

According to an embodiment, said at least one cannabinoid is selected from the group consisting of CBD, THC and CBN, wherein said at least one terpene is selected from the group consisting of terpinene, linalool, pinene, caryophyllene, geraniol, limonene, bisabolol and combinations thereof and wherein said condition or symptom thereof comprises poor immune response.

According to an embodiment, said at least one cannabinoid is selected from the group consisting of CBD, THC, THCa and CBN, wherein said at least one terpene is selected from the group consisting of myrcene, pinene, nerolidol, linalool, humulene, caryophyllene, limonene, bisabolol, eucalyptol, terpineol, geraniol, boeneol, sabinene, ocimene and combinations thereof and wherein said condition or symptom thereof is selected from the group consisting of muscle tension, cramps, pain and combinations thereof.

According to an embodiment, said at least one cannabinoid is selected from the group consisting of CBD, THC, and THCa, wherein said at least one terpene is selected from the group consisting of linalool, geraniol, humulene, bisabolol, fenchol, linalool, limonene, terpineol, pinene, caryophyllene, myrcene, terpinene, borneol, sabinene, ocimene and combinations thereof and wherein said condition or symptom thereof comprises nausea and/or vomiting.

According to an embodiment, said at least one terpene comprises at least two, at least three or at least four terpenes. In such embodiments, said at least two, at least three or at least four terepenes are present in the composition at a concentration of at least 20 wt % based on the weight of the total amount of terpenes in the composition.

According to an aspect of some embodiments of the present invention, there is provided a first-aid kit comprising a composition comprising at least one cannabinoid; at least one terpene selected from the group consisting of Pinene, Myrcene, Caryophyllene, Humulene, Nerolidol, Bisabolol, Linalool, Fenchol, Limonene, Terpinene, Terpineol, Eucalyptol, geraniol and combinations thereof, wherein said at least one terpene is present in the composition at a concentration of at least about 40 wt %, based on the weight of the total amount of terpenes in the composition; and at least 5% by weight of a non-cannabinoid, non-terpene carrier.

### DETAILED DESCRIPTION OF THE INVENTION

Unless indicated otherwise the term “trauma” as used herein refers to any deeply distressing or disturbing experience, including motor vehicle collisions, gunshot, terrorist attack, falls etc.

As used herein the term “treating” includes preventing, curing, ameliorating, mitigating, and reducing the instances or severity of a condition or a symptom thereof.

As used herein, the term “preventing” with regard to an occurrence of a condition or symptom thereof refers to stopping or reducing the condition or symptom thereof from occurring due to exposure to a stressful event.

As used herein the term “reducing” refers to reducing the duration or intensity of the condition or symptom by at least 10% as compared to the duration or intensity occurring in the absence of administration of the compositon of the present invention. According to some embodiments, the duration or intensity of the condition or symptom may be determined by clinical assays, as known in the art. According to some embodiments, the duration or intensity of the condition or symptom may be determined according to patient-reported parameters.

Unless indicated otherwise, percent is weight percent and ratio is weight/weight ratio. Unless indicated otherwise, weight ratio means the ratio between weight content, e.g. in an aqueous solution containing 20% solute and 80% water, the solute to water weight ratio is 20:80 or 1:4.

Unless indicated otherwise, the term cannabinoid as used herein refers to a compound that affects the endocannabinoid system. Cannabinoids are agonists or antagonists to receptors in the endocannabinoid system.

As used herein, the term THC refers to THCa (tetrahydrocannabiniolic acid) and/or to THC (tetrahydrocannabiniol) unless indicated otherwise. As used herein, the term CBD refers to CBDa (cannabidiolic acid) and/or to CBD (cannabidiol) unless indicated otherwise. As used herein, the term CBG refers to CBGa (cannabigerolic acid) and/or to CBG (cannabigerol) unless indicated otherwise. As used herein, the term CBN refers to CBNa (Cannabinolic acid) and/or to CBN (cannabinol) unless indicated otherwise. As used herein, the term CBC refers to CBCa (cannabichromenic acid) and/or to CBC (Cannabichromene) unless indicated otherwise. As used herein, the term CBL refers to CBLa (Cannabicycol acid) and/or to CBL (Cannabicyclol) unless indicated otherwise. As used herein, the term THCV refers to THCVa (tetrahydrocannabivarin acid) and/or to THCV (tetrahydrocannabivarin) unless indicated otherwise. As used herein, the term CBDV refers to CBDVa (cannabigerovarin acid) and/or to CBDV (cannabidivarin) unless indicated otherwise.

As used herein, the term cellulose refers to cellulose, hemicellulose and their combinations. As used herein, the term glycol refers to any glycol, including ethylene glycol, polyethylene glycol, propylene glycol and polypropylene glycol. As used herein, the term chlorophyll refers to chlorophyll and degradation products thereof.

As used herein, “terpene” refers to both terpenes and terpenoids.

As used herein and unless indicated otherwise, the term “terpenes/cannabinoids (or terpenes to cannabinoids) weight/weight ratio” means the weight ratio between the combined amount of terpenes and the combined amount of cannabinoids.

The particulars shown herein are by way of example and for purposes of illustrative discussion of the various embodiments of the present invention only and are presented in the cause of providing what is believed to be the most useful and readily understood description of the principles and conceptual aspects of the invention. In this regard, no attempt is made to show details of the invention in more detail than is necessary for a fundamental understanding of the invention, the description making apparent to those skilled in the art how the several forms of the invention may be embodied in practice.

The present invention will now be described by reference to more detailed embodiments. This invention may, however, be embodied in different forms and should not be construed as limited to the embodiments set forth herein. Rather, these embodiments are provided so that this disclosure will be thorough and complete, and will fully convey the scope of the invention to those skilled in the art.

Unless otherwise defined, all technical and scientific terms used herein have the same meaning as commonly understood by one of ordinary skill in the art to which this invention belongs. The terminology used in the description of the invention herein is for describing particular embodiments only and is not intended to be limiting of the invention. As used in the description of the invention and the appended claims, the singular forms “a,” “an,” and “the” are intended to include the plural forms as well, unless the context clearly indicates otherwise.

Unless otherwise indicated, all numbers expressing quantities of ingredients, reaction conditions, and so forth used in the specification and claims are to be understood as being modified in all instances by the term “about.” Accordingly, unless indicated to the contrary, the numerical parameters set forth in the following specification and attached claims are approximations that may vary depending upon the desired properties sought to be obtained by the present invention. At the very least, and not as an attempt to limit the application of the doctrine of equivalents to the scope of the claims, each numerical parameter should be construed in light of the number of significant digits and ordinary rounding approaches.

Notwithstanding that the numerical ranges and parameters setting forth the broad scope of the invention are approximations, the numerical values set forth in the specific examples are reported as precisely as possible. Any numerical value, however, inherently contains certain errors necessarily resulting from the standard deviation found in their respective testing measurements. Every numerical range given throughout this specification will include every narrower numerical range that falls within such broader numerical range, as if such narrower numerical ranges were all expressly written herein.

As used herein, the terms “comprising”, “including”, “having” and grammatical variants thereof are to be taken as specifying the stated features, integers, steps or components but do not preclude the addition of one or more additional features, integers, steps, components or groups thereof. These terms encompass the terms “consisting of” and “consisting essentially of”.

Additional advantages of the invention will be set forth in part in the description which follows, and in part will be obvious from the description, or may be learned by practice of the invention. It is to be understood that both the foregoing general description and the following detailed description are exemplary and explanatory only and are not restrictive of the invention, as claimed.

According to an embodiment, there is provided a terpene-enriched cannabinoid composition comprising at least one cannabinoid in a specific amount, at least one primary terpene in a specific amount, wherein the at least one primary terpene forms at least 40% by weight of a total amount of terpenes in the composition, at least 5% by weight of a non-cannabinoid, non-terpene carrier; and optionally at least two secondary terpenes; and wherein the non-cannabinoid, non-terpene carrier comprises cellulose and a weight/weight ratio of the total amount of terpenes to total cannabinoids in the composition is from about 0.1:1 to about 1:1, or; (b) wherein the non-cannabinoid, non-terpene carrier comprises less than 5% by weight cellulose and a weight/weight ratio of the total amount of terpenes to total cannabinoids in the composition is from about 0.05:1 to about 1:1.

Further provided is a cannabinoid composition treating conditions and/or symptoms associated with a stressful event comprising (i) at least one cannabinoid in a specific amount, (ii) at least one primary terpene in a specific amount wherein the at least one primary terpene forms at least 40% by weight of a total amount of terpenes in the composition, (iii) at least 5% by weight of a non-cannabinoid, non-terpene, carrier, (iv) optionally at least two secondary terpenes; and (a) wherein the non-cannabinoid, non-terpene carrier comprises cellulose and the terpenes to cannabinoids weight/weight ratio in the composition is from about 0.1:1 to about 1:1, or (b) wherein the non-cannabinoid, non-terpene carrier comprises less than 5% by weight cellulose and the terpenes to cannabinoids weight/weight ratio in the composition is from about 0.05:1 to about 1:1.

According to an embodiment, the composition has an therapeutic effect in treating conditions and/or symptoms associated with a stressful event, wherein the therapeutic effect is an enhanced therapeutic effect compared with that of a composition comprising the same cannabinoids amounts and one half, one third, one quarter, or one fifth the amount of the at least one primary terpene. According to one embodment, the enhanced therapeutic effect is compared with that of a composition comprising the same cannabinoids amount and one fifth the amout of the at least one primary terpene.

According to an embodiment, the compostion comprises less than 5% by weight glycol

According to an embodiment, the composition has a water concentration of less than 20% by weight.

According to an embodiment, the conditions and/or symptoms are selected from the group consisting of stress, distress, acute anxiety, trauma-related anxiety, depression, acute stress, post traumatic stress, acute pain, trauma-related pain, neuronal injury, bacterial infection, muscle tension, inflammation and combination thereof. According to an embodiment, the therapeutic effect increases immune response.

According to an embodiment, the stressful event includes surgery, dental treatment, unpleasant and/or painful medical procedure, sport injury, battlefield injury, traumatic injury, sudden physical injury, sprain, joint dislocation, bone fracture, childbirth, stressful examination, stressful social event, trauma, major trauma, psychological trauma and combination thereof.

According to an embodiment, the therapeutic effect treats acute or sudden onset conditions and/or symptoms.

According to an embodiment, the composition comprises a first aid treatment.

According to an embodiment, the therapeutic effect treats delayed conditions and/or symptoms.

According to an embodiment, the composition comprises at least two cannabinoids, at least three, at least four or at least five. According to an embodiment, the content of each cannabinoid in the composition is at least 10 parts per million (ppm). As known in the art, cannabinoids have an acid form and a non-acid form (which is also referred to as decarboxylated form, since it can be generated by decarboxylating the acid form). The acid form is indicated herein by the letter (a) at the end of the cannabinoid acronym, e.g. tetrahydrocannabiniolic acid is indicated as THCa, while the decarboxylated form is THC. According to an embodiment, the cannabinoids are selected from the group consisting of tetrahydrocannabiniol in acid or decarboxylated form (THCa or THC, respectively), cannabidiol in acid or decarboxylated form (CBDa or CBD, respectively), cannabigerol in acid or decarboxylated form (CBGa or CBG, respectively), cannabichromene in acid or decarboxylated form (CBCa or CBC, respectively) tetrahydrocannabivarin in acid or decarboxylated form (THCVa or THCV, respectively), Cannabidivarin in acid or decarboxylated form (CBDVa or CBDV respectively) Cannabinol in acid or decarboxylated form (CBNa or CBN, respectively), Cannabicyclol in acid or decarboxylated form (CBLa or CBL, respectively). As used herein, the term “THC” refers to THCa (tetrahydrocannabiniolic acid) and/or to THC (tetrahydrocannabiniol) unless indicated otherwise. As used herein, the term “CBD” refers to CBDa (teannabidiolic acid) and/or to CBD (cannabidiol) unless indicated otherwise. Thus, the term “CBD to THC ratio” may mean “CBD to THC ratio”, “CBDa to THC ratio”, “CBD to THCa ratio”, “CBDa to THCa ratio”, “CBD to THC+THCa ratio”, “CBDa to THC+THCa ratio”, “CBD+CBDa to THC ratio”, “CBD+CBDa to THCa ratio” or “CBD+CBDa to THC+THCa ratio”. As used herein, the term “CBG” refers to CBGa (cannabigerolic acid) and/or to CBG (cannabigerol) unless indicated otherwise. As used herein, the term “CBN” refers to CBNa (Cannabinolic acid) and/or to CBN (cannabinol) unless indicated otherwise. As used herein, the term “CBC” refers to CBCa (cannabichromenic acid) and/or to CBC (Cannabichromene) unless indicated otherwise.

According to an embodiment, at least one of the cannabinoids is in acid form. According to an embodiment, at least one of the cannabinoid is at least partially in decarboxylated form. According to an embodiment, at least 50% of the cannabinoid is in decarboxylated form, at least 60%, at least 70%, at least 80% or at least 90%.

According to an embodiment, the composition comprises THC and/or THCa. According to an embodiment, the composition comprises CBD and/or CBDa. According to an embodiment, the composition comprises THC and/or THCa at a content of less than 1%, less than 0.8%, less than 0.6%, less than 0.4% or less than 0.2%. According to an embodiment, the composition comprises both CBD and/or CBDa and THC and/or THCa and the weight/weight ratio between CBD and/or CBDa and THC and/or THCa ((CBD+CBDa)/(THC+THCa)) is at least 10, at least 15, at least 20, at least 25 or at least 30. According to an embodiment, the composition comprises CBG and/or CBGa. According to an embodiment, the composition comprises CBN and/or CBNa. According to an embodiment, the composition comprises CBC and/or CBCa. According to an embodiment, the product comprises CBL and/or CBLa. According to an embodiment, the product comprises THCV and/or THCVa. According to an embodiment, the product comprises CBDV and/or CBDVa.

According to an embodiment, the composition comprises at least 2% by weight carrier, at least 3%, at least 5%, at least 10%, at least 15%, at least 20%, at least 25%, at least 30%, at least 35% or at least 40% by weight. Any compound other than cannabinoids and terpenes is a suitable carrier. According to an embodiment, the carrier is selected from the group consisting of vegetable oils, e.g. coconut oil, olive oil or sesame oil, pharmaceutical excipients, honey, bees wax, cellulose and combinations thereof. As used herein, the term “cellulose” refers to cellulose, hemicellulose and their combinations.

According to an embodiment, the composition comprises less than 5% by weight glycol, less than 4%, less than 3%, less than 2%, or less than 1%, by weight glycol. As used herein, the term “glycol” refers to any glycol, including ethylene glycol, polyethylene glycol, propylene glycol and polypropylene glycol.

According to an embodiment, the composition comprises water and water content is less than 20%, less than 15%, less than 12%, less than 10%, or less than 8%. According to an embodiment, water content is at least 1% by weight, at least 2%, at least 3%, at least 4% or at least 5%.

According to an embodiment, the composition comprises a primary terpene and optionally at least two secondary terpenes, at least three, at least four or at least five. The term “terpene”, as used herein, refers to both terpenes and terpenoids. As used here, the term “primary terpene” refers to a terpene that forms at least 20% by weight of the total amount of terpenes in the composition, at least 30%, at least 40%, at least 50%, at least 60%, at least 70% or at least 80%. In some preferred embodiments, the primary terpene forms at least 40% by weight of the total amount of terpenes in the composition. According to an embodiment, the composition comprises multiple (e.g. two or three) terpenes, each one of which forms at least 20% by weight of the total amount of terpenes in the composition and each one of these terpenes is considered a primary terpene. As used here, the term “secondary terpene” refers to a terpene that forms at least 10 parts per million (ppm) of the composition. According to an embodiment, the content of the primary terpene in the composition is at least 2 times greater than that of any secondary terpene, at least 4, at least 6, at least 8, at least 10, at least 15, at least 20, at least 25, or at least 30 times greater.

As used herein, “terpenes/cannabinoids (or terpenes to cannabinoids) weight/weight ratio” means the weight ratio between the combined amount of terpenes and the combined amount of cannabinoids. According to an embodiment, the composition comprises at least 5% by weight cellulose and the terpenes to cannabinoids weight/weight ratio in the composition is in the range between from about 0.1 and about 1.0. According to an embodiment, the ratio is greater than 0.1, greater than 0.15, greater than 0.2, greater than 0.25, greater than 0.3, greater than 0.35, greater than 0.4, greater than 0.45, greater than 0.5, greater than 0.55, greater than 0.6, greater than 0.65, greater than 0.7, greater than 0.75, greater than 0.8, greater than 0.85, greater than 0.9, greater than 0.95, greater than 1, greater than 1.2, greater than 1.5, greater than 2.0, greater than 3 or greater than 5. According to an embodiment, the ratio is less than 0.9, less than 0.8, less than 0.7, less than 0.6, less than 0.5, less than 0.4, less than 0.3, less than 0.2 or less than 0.15. According to an embodiment, the composition comprising more than 5% cellulose is selected from the group consisting of cannabis plant material, e.g. cannabis buds or cannabis trim, ground forms thereof, plant material preparation for vaporizers and cannabis cigarette. According to an embodiment, the product comprises a dried cannabis plant material.

According to another embodiment, the non-cannabinoid, non-terpene carrier comprises less than 5% cellulose and terpenes to cannabinoids weight/weight ratio in the composition is in the range between about 0.05 and about 1.0. According to an embodiment, the ratio is greater than 0.1, greater than 0.15, greater than 0.2, greater than 0.25, greater than 0.3, greater than 0.35, greater than 0.4, greater than 0.45, greater than 0.5, greater than 0.55, greater than 0.6, greater than 0.65, greater than 0.7, greater than 0.75, greater than 0.8, greater than 0.85, greater than 0.9, greater than 0.95, greater than 1, greater than 1.2, greater than 1.5, greater than 2.0, greater than 3 or greater than 5. According to an embodiment, the ratio is less than 0.9, less than 0.8, less than 0.7, less than 0.6, less than 0.5, less than 0.4, less than 0.3, less than 0.2, less than 0.15 or less than 0.1. According to an embodiment, the composition comprising less than 5% cellulose is selected from the group consisting of cannabis trichomes, cannabis extracts and products thereof, such as tablets, gel capsules, medical patches, vaporizer liquids and suppositories.

According to an embodiment, the primary terpene, the secondary terpenes or both are selected from the group consisting of pinene, limonene, linalool, caryophyllene, myrcene, humulene, borncol, cucalyptol, terpineol, nerolidol, phytol, geraniol, bisabolol, camphene, beta-amyrin, thujone, citronellol, pulegone, cycloartenol, cymene, sabinene, carene, terpinene, fenchol, isopulegol, guaiol, phellandrene, eudesmol, ocimene, cardinene, elemene, gurjunene, farnesene, cardinol, elemene, friedelin, carvacrol, eugenol, camphor, menthol, iso-menthone, neral, gerial, viridiflorol, germacrene, thymol, Menth-2-en-1-ol, farensol, carotol, myrtenol isomers thereof and combinations thereof. According to an embodiment, the primary terpene the secondary terpenes or both, are selected from the group consisting of pinene, limonene, linalool, caryophyllene, myrcene, terpinene, terpincol and combinations thereof. According to an embodiment, at least one of the terpenes is a-cyclic. According to an embodiment, at least one of the terpenes is cyclic. According to an embodiment, at least one of the terpenes is not found in cannabis buds or is present there at less than 0.2%, less than 0.1%, less than 0.05% or less than 0.02%. Such terpene is referred to as “non-cannabis terpene”. According to an embodiment, the terpene-enriched cannabinoid composition comprises the non-cannabis terpene at a concentration of at least 0.2%, at least 0.5%, least 0.8%, at least 1%, least 1.5%, at least 2%, at least 3%, least 4%, at least 5%, at least 8% or at least 12%.

According to an embodiment, the primary terpene is selected from the group consisting of pinene, limonene, linalool, caryophyllene, myrcene, humulene, borneol, eucalyptol, terpineol, nerolidol, phytol, geraniol, bisabolol, camphene, beta-amyrin, thujone, citronellol, pulegone, cycloartenol, cymene, sabinene, carene, terpinene, fenchol, isopulegol, guaiol, phellandrene, eudesmol, ocimene, cardinene, elemene, gurjunene, farnesene and combinations thereof. According to an embodiment, the primary terpene is selected from the group consisting of pinene, limonene, linalool, caryophyllene, myrcene and humulene. According to an embodiment, the primary terpene is a non-cannabis terpene.

According to an embodiment, the terpenes comprise at least one monoterpene selected from the group consisting of limonene, myrcene, pinene, linalool, geraniol, terpinolene camphene and isomers thereof. According to an embodiment, the terpenes comprise at least one sesquiterpene selected from the group consisting of nerolidol, caryophyllene, farnesene, zingiberene, vetivazulene, guaiazulene, longifolene, copaene, patchoulol humulene and isomers thereof. According to an embodiment, the terpenes comprise at least one diterpene selected from the group consisting of phytol, retinal, retinol, phytane, cembrene, sclarene, labdane, abietane, texadiene, stemarene, stemoden and isomers thereof. According to an embodiment, the terpenes comprise at least one hydroxy-terpene selected from the group consisting of nerolidol, geraniol, linalool, phytol and isomers thereof. As used herein, the term “hydroxy-terpene” refers to a terpene carrying a hydroxyl function.

According to an embodiment, at least one of the terpenes is a monoterpene, at least one of the terpenes is a sesquiterpene and the monoterpenes to sesquiterpenes weight/weight ratio (i.e. the weight ratio between the total amount of monoterpenes and the total amount of the sesquiterpenes) is greater than 1.5 greater than 2, greater than 2.5, greater than 3, greater than 3.5, greater than 4, greater than 4.5, greater than 5, greater than 6, greater than 7, greater than 8, greater than 9, greater than 10, greater than 15, or greater than 20.

According to an embodiment, at least one of the terpenes is a monoterpene, at least one of the terpenes is a diterpene and the monoterpenes to diterpenes weight/weight ratio (i.e. the weight ratio between the total amount of monoterpenes and the total amount of the diterpenes) is greater than 5, greater than 6, greater than 7, greater than 8, greater than 9, greater than 10, greater than 12, greater than 14, greater than 16, greater than 18, greater than 20, greater than 25, or greater than 30.

According to an embodiment, at least one of the terpenes carries no hydroxyl group, at least one of the terpenes carries hydroxyl group and the non-hydroxy-terpenes to hydroxyl-terpenes weight/weight ratio (i.e. the weight ratio between the total amount of non-hydroxy-terpenes and the total amount of the hydroxy-terpenes) is greater than 1.5, greater than 2, greater than 2.5, greater than 3, greater than 3.5, greater than 4, greater than 4.5, greater than 5, greater than 6, greater than 7, greater than 8, greater than 9, greater than 10, greater than 15, or greater than 20.

According to an embodiment, terpenes form at least 20% by weight of the composition, at least 30%, at least 40%, at least 50%, at least 60% or at least 70% of the weight of the total composition.

According to an embodiment, the composition is liquid at 30° C. According to an embodiment, the composition is a suspension at 30° C. According to an embodiment, the composition is essentially clear of haze or suspended solids at 30° C.

According to an embodiment, the composition comprises cannabis plant material. According to an embodiment, the composition comprises cannabis bud. According to an embodiment, the cannabis bud forms at least 20% of the composition, at least 30%, at least 40%, at least 50%, at least 60%, at least 70% or at least 80%.

According to an embodiment the product comprises tetrahydrocannabinol (THC) and/or tetrahydrocannabinolic acid (THCa), wherein THC and/or THCa is in a total concentration of at least 1% by weight, at least 2%, at least 3%, at least 4%, at least 5%, at least 6%, at least 8%, at least 10%, at least 12%, at least 14%, at least 16%, at least 18%, or at least 20%, at least 30%, at least 40%, at least 50%, at least 60%, at least 70%, at least 80%, or at least 90% by weight.

According to an embodiment, the product comprises cannabidiol (CBD) and/or cannabidiolic acid (CBDa), wherein CBD and/or CBDa is in a total concentration of at least 1% by weight, at least 2%, at least 3%, at least 4%, at least 5%, at least 6%, at least 8%, at least 10%, at least 12%, at least 14%, at least 16%, at least 18% or at least 20%, at least 30%, at least 40%, at least 50%, at least 60%, at least 70%, at least 80%, or at least 90% by weight.

According to an embodiment, the product comprises tetrahydrocannabinol (THC) and/or tetrahydrocannabinolic acid (THCa) and cannabidiol (CBD) and/or cannabidiolic acid (CBDa), wherein, THC and/or THCa is in a total concentration of at least 2.5% by weight, and CBD and/or CBDa is in a total concentration of at least 2.5% by weight; THC and/or THCa in a total concentration of at least 3% by weight, and CBD and/or CBDa in a total concentration of at least 3% by weight; THC and/or THCa in a total concentration of at least 4% by weight, and CBD and/or CBDa in a total concentration of at least 4% by weight; THC and/or THCa in a total concentration of at least 5% by weight, and CBD and/or CBDa in a total concentration of at least 5% by weight; THC and/or THCa in a total concentration of at least 8% by weight, and CBD and/or CBDa in a total concentration of at least 8% by weight; THC and/or THCa in a total concentration of at least 10% by weight, and c CBD and/or CBDa in a total concentration of at least 10% by weight.

According to a related embodiment, said product comprises cannabigerol (CBG) and/or cannabigerol acid (CBGa) in a total concentration of at least 0.1% by weight, at least 0.5%, at least 1%, at least 2%, at least 3%, at least 4%, or at least 5%, at least 6%, at least 8%, at least 10%, at least 12%, at least 14%, at least 16%, at least 18% or at least 20%, at least 30%, at least 40%, at least 50%, at least 60%, at least 70%, at least 80%, or at least 90% by weight. According to a related embodiment, said product comprises cannabinol (CBN) and/or cannabinol acid (CBNa) in a total concentration of at least 0.1% by weight, at least 0.5%, at least 1%, at least 2%, at least 3%, at least 4%, or at least 5%, at least 6%, at least 8%, at least 10%, at least 12%, at least 14%, at least 16%, at least 18% or at least 20%, at least 30%, at least 40%, at least 50%, at least 60%, at least 70%, at least 80%, or at least 90% by weight. According to a related embodiment, said product comprises cannabichromene (CBC) and/or cannabichromenic acid (CBCa) in a total concentration of at least 0.1% by weight, at least 0.5%, at least 1%, at least 2%, at least 3%, at least 4%, or at least 5%, at least 6%, at least 8%, at least 10%, at least 12%, at least 14%, at least 16%, at least 18% or at least 20%, at least 30%, at least 40%, at least 50%, at least 60%, at least 70%, at least 80%, or at least 90% by weight. According to a related embodiment, said product comprises cannabicyclol (CBL) and/or cannabicyclol acid (CBLa) in a total concentration at least 0.1% by weight, at least 0.5%, at least 1%, at least 2%, at least 3%, at least 4%, or at least 5%, at least 6%, at least 8%, at least 10%, at least 12%, at least 14%, at least 16%, at least 18% or at least 20%, at least 30%, at least 40%, at least 50%, at least 60%, at least 70%, at least 80%, or at least 90% by weight. According to a related embodiment, said product comprises tetrahydrocannabivarin (THCV), and/or tetrahydrocannabivarin acid (THCVA) in a total concentration at least 0.1% by weight, at least 0.5%, at least 1%, at least 2%, at least 3%, at least 4%, or at least 5%, at least 6%, at least 8%, at least 10%, at least 12%, at least 14%, at least 16%, at least 18% or at least 20%, at least 30%, at least 40%, at least 50%, at least 60%, at least 70%, at least 80%, or at least 90% by weight. According to a related embodiment, said product comprises cannabidivarin (CBDV) and/or cannabigerovarin acid (CBGVA) in a total concentration at least 0.1% by weight, at least 0.5%, at least 1%, at least 2%, at least 3%, at least 4%, or at least 5%, at least 6%, at least 8%, at least 10%, at least 12%, at least 14%, at least 16%, at least 18% or at least 20%, at least 30%, at least 40%, at least 50%, at least 60%, at least 70%, at least 80%, or at least 90% by weight.

According to an embodiment, the product comprises less than 5% by weight glycol, less than 4%, less than 3%, less than 2%, or less than 1%, by weight glycol. As used herein, the term glycol refers to any glycol, including ethylene glycol, polyethylene glycol, propylene glycol and polypropylene glycol.

According to an embodiment, the product comprises water and water content is less than 30% by weight, less than 25%, less than 20%, less than 15%, less than 12%, less than 10%, or less than 8%. According to an embodiment, water content is at least 1% by weight, at least 2%, at least 3%, at least 4% or at least 5% by weight. According to an embodiment, the product comprises water and water content is more than 50%, more than 60%, more than 70%, more than 80%, more than 90%, or more than 95%.

According to an embodiment, the product comprises chlorophyll. According to an embodiment, the product comprises at least 0.5% chlorophyll, at least 1, % at least 5%, at least 10%, at least 15%, at least 20% chlorophyll. As used herein, the term chlorophyll refers to chlorophyll and degradation products thereof. According to an embodiment, the product comprises at least one flavonoid. According to an embodiment, the product comprises at least two, at least three, at least four, or at least five flavonoids. According to an embodiment, the product comprises at least one of bergamottin, apigenin, amentoflavone, quercetin and piperine. According to an embodiment, the product comprises at least two, at least three, or at least four of bergamottin, apigenin, amentoflavone, quercetin and piperine. According to an embodiment, the product comprises bergamottin. According to an embodiment, the product comprises apigenin. According to an embodiment, the product comprises amentoflavone. According to an embodiment, the product comprises quercetin. According to an embodiment, the product comprises piperine.

According to an embodiment, the composition comprises an additive selected from the group consisting of antioxidants, emulsifiers and texturizers vegetable oils, plant extracts, honey, pharmaceutical excipients, sucrose, glucose and fructose, pharmaceutical excipients and combinations thereof. According to an embodiment, the composition comprises a surfactant selected from the group consisting of phospholipids, glycerides, glycolipids and combinations thereof. According to an embodiment, the composition further comprises a food-approved texturizer. According to an embodiment, the composition further comprises at least 10 ppm ethanol. According to an embodiment, the composition further comprises at least one of vitamin C, vitamin E, polyunsaturated fatty acids, beeswax and coconut oil. According to an embodiment, the product further comprises a sweetener.

According to an embodiment, the composition is selected from the group consisting of cannabis tablets, cannabis gel capsules, cannabis medical patches, cannabis cigarettes and cannabis vaporizer liquids, cannabis suppositories, cannabis candies, cannabis drinks and cannabis baked products.

According to an embodiment, the composition results in an increased therapeutic effect compared with that of a composition comprising the same cannabinoids amounts and a smaller amount of the primary terpene, e.g. one half, one third, one quarter, one fifth of that amount. According to various embodiment, the increased therapeutic effect has various forms, e.g. a shorter onset time, increased magnitude, extended duration, reduced dosages, reduced secondary adverse symptoms, reduced frequency of conditions and/or symptoms, reduced severity of conditions and/or symptoms, reduced consumption of other drugs and combinations thereof. According to an embodiment, the increased therapeutic effect comprises a shorter onset time, or differently put an earlier effect, which is important particularly in cases of sublingual and topical delivery and in cases where a rapid effect is desired, as in treating trauma patients. According to an embodiment, the increased therapeutic effect comprises extended duration of the therapeutic effect, for example an extended time of pain relief. According to an embodiment, the increased therapeutic effect comprises increased magnitude of the therapeutic effect, enabling achieving a desired therapeutic effect on administering smaller doses of cannabinoids, saving thereby on cost. According to an embodiment, the increased therapeutic effect comprises using smaller doses of cannabinoids and still achieving at least the same beneficial result. According to an embodiment, the increased therapeutic effect comprises reduction of secondary adverse symptoms, e.g. adverse symptoms of the main illness, of ones of another illness and/or ones related to administered the composition or other drugs. According to an embodiment, the increased therapeutic effect comprises reduced frequency of the conditions and/or symptoms. According to an embodiment, the increased therapeutic effect comprises reduced severity of the conditions and/or symptoms.

According to an embodiment, the primary terpene and the cannabinoids are present in specific amounts, and the onset time of the therapeutic effect, as measured by methods known in the art, is at least 20% shorter than that of a composition comprising the same cannabinoids amounts and one half, one third, one quarter or one fifth the amount of the primary terpene, at least 30%, at least 40%, at least 50% or at least 60% shorter. According to an embodiment, the primary terpene, secondary terpenes and the cannabinoids are present in specific amounts, and the onset time of the therapeutic effect is at least 20% shorter than that of a composition comprising the same cannabinoids amounts, same secondary terpene amounts and one fifth the amount of the primary terpene, at least 30%, at least 40%, at least 50% or at least 60% shorter. Shorter onset time, or differently put an earlier effect, is important particularly in cases of sublingual and topical delivery and in cases where a rapid effect is desired, as in treating pain.

According to an embodiment, the primary terpene and the cannabinoids are present in specific amounts, and the onset time of the therapeutic effect is at least 20% longer than that of a composition comprising the same cannabinoids amounts and one fifth the amount of the primary terpene, at least 30%, at least 40%, at least 50% or at least 60% longer. According to an embodiment, the primary terpene, secondary terpenes and the cannabinoids are present in specific amounts, and the onset time of the therapeutic effect is at least 20% longer than that of a composition comprising the same cannabinoids amounts, same secondary terpene amounts and one half the amount of the primary terpene, at least 30%, at least 40%, at least 50% or at least 60% longer. According to an embodiment, compositions of delayed onset time are used in combination with shorter onset time to reach a sustained release effect.

According to an embodiment, the primary terpene and the cannabinoids are present in specific amounts, and the magnitude of the therapeutic effect, as measured by methods known in the art, is at least 20% greater compared with that of a composition comprising the same cannabinoids amounts and one half, one third, one quarter or one fifth the amount of the primary terpene, at least 30%, at least 40%, at least 50% or at least 60% greater. According to an embodiment, the primary terpene, secondary terpenes and the cannabinoids are present in specific amounts, and the magnitude of the therapeutic effect is at least 20% greater than that of a composition comprising the same cannabinoids amounts, same secondary terpene amounts and one fifth the amount of the primary terpene, at least 30%, at least 40%, at least 50% or at least 60% greater. Without wishing to be limited by any particular theory, such increased magnitude of the therapeutic effect may indicate increased bioavailability. Such increased magnitude enables achieving a desired therapeutic effect on administering smaller doses of cannabinoids, saving thereby on cost.

According to an embodiment, the primary terpene and the cannabinoids are present in specific amounts, and the duration of the therapeutic effect, as measured by methods known in the art, is at least 20% longer compared with that of a composition comprising the same cannabinoids amounts and one half, one third, one quarter or one fifth the amount of the primary terpene, at least 30%, at least 40%, at least 50% at least 60%.at least 70%, at least 80%, at least 90% or at least 100% longer. According to an embodiment, the primary terpene, secondary terpenes and the cannabinoids are present in specific amounts, and the duration of the therapeutic effect is at least 20% longer than that of a composition comprising the same cannabinoids amounts, same secondary terpene amounts and one fifth the amount of the primary terpene, at least 30%, at least 40%, at least 50%, at least 60%, at least 70%, at least 80%, at least 90% or at least 100% longer.

According to an embodiment, the primary terpene and the cannabinoids are present in specific amounts, and the frequency of the conditions and/or symptoms, as measured by methods known in the art, is at least 20% smaller compared with that of a composition comprising the same cannabinoids amounts and one half, one third, one quarter or one fifth the amount of the primary terpene, at least 30%, at least 40%, at least 50% at least 60%.at least 70%, at least 80%, or at least 90% smaller. According to an embodiment, the primary terpene, secondary terpenes and the cannabinoids are present in specific amounts, and the frequency of the conditions and/or symptoms, as measured by methods known in the art, is at least 20% smaller than that of a composition comprising the same cannabinoids amounts, same secondary terpene amounts and one fifth the amount of the primary terpene, at least 30%, at least 40%, at least 50%, at least 60%.at least 70%, at least 80%, at least 90% or at least 100% longer.

According to an embodiment, the primary terpene and the cannabinoids are present in specific amounts, and the severity of the conditions and/or symptoms, as measured by methods known in the art, is at least 20% smaller compared with that of a composition comprising the same cannabinoids amounts and one half, one third, one quarter or one fifth the amount of the primary terpene, at least 30%, at least 40%, at least 50% at least 60%.at least 70%, at least 80%, or at least 90% smaller. According to an embodiment, the primary terpene, secondary terpenes and the cannabinoids are present in specific amounts, and the severity of the conditions and/or symptoms, as measured by methods known in the art, is at least 20% smaller than that of a composition comprising the same cannabinoids amounts, same secondary terpene amounts and one fifth the amount of the primary terpene, at least 30%, at least 40%, at least 50%, at least 60%.at least 70%, at least 80%, at least 90% or at least 100% longer.

According to an embodiment, the primary terpene and the cannabinoids are present in specific amounts, and the reduced requirement for the composition, as measured by methods known in the art, is a reduction at least 20% in the dosage required to obtain the same therapeutic effect as that obtained by administration of a composition comprising the same cannabinoids amounts and one half, one third, one quarter or one fifth the amount of the primary terpene, at least 30%, at least 40%, at least 50% at least 60%.at least 70%, at least 80%, or at least 90% reduction. According to an embodiment, the primary terpene, secondary terpenes and the cannabinoids are present in specific amounts, and the reduced requirement for the composition, as measured by methods known in the art, is a reduction at least 20% in the dosage required to obtain the same therapeutic effect as that obtained by administration of a composition comprising the same cannabinoids amounts, same secondary terpene amounts and one fifth the amount of the primary terpene, at least 30%, at least 40%, at least 50%, at least 60%.at least 70%, at least 80%, at least 90% or at least 100% reduction.

According to an embodiment, the primary terpene and the cannabinoids are present in specific amounts, and the reduced amount or frequency of other drugs required, as measured by methods known in the art, is a reduction at least 20% in the amount or frequency of other drugs required to obtain the same therapeutic effect as that obtained by administration of a composition comprising the same cannabinoids amounts and one half, one third, one quarter or one fifth the amount of the primary terpene, at least 30%, at least 40%, at least 50% at least 60%.at least 70%, at least 80%, or at least 90% reduction. According to an embodiment, the primary terpene, secondary terpenes and the cannabinoids are present in specific amounts, and the reduced amount or frequency of other drugs, as measured by methods known in the art, is a reduction at least 20% in the amount or frequency required to obtain the same therapeutic effect as that obtained by administration of a composition comprising the same cannabinoids amounts, same secondary terpene amounts and one fifth the amount of the primary terpene, at least 30%, at least 40%, at least 50%, at least 60%.at least 70%, at least 80%, at least 90% or at least 100% reduction.

According to an embodiment, the therapeutic effect is generated while administering the composition in a vaporizer. According to an embodiment, the therapeutic effect is generated while administering the composition sublingually. According to an embodiment, the therapeutic effect is generated while applying the composition, topically.

According to an embodiment, the composition comprises at least one of CBD, THC, CBDa, THCa, CBG, CBC and CBN, CBL, THCV and CBDV, and primary terpene is selected from the group consisting of linalool, limonene, cucalyptol, myrcene, caryophyllene, humulene, camphene, borneol, terpineol, pinene, terpinene, citronellol, amyrin, cycloartenol, camphor, sabinene, geraniol, guaiol, B-Eudesmol, menthol, bisabolol, fenchol, nerolidol, phellandrene, cymene, farnesene, citral and combinations thereof. According to an embodiment, the composition comprises CBD. According to an embodiment, the composition comprises THC. According to an embodiment, the composition comprises CBDa. According to an embodiment, the composition comprises THCa. According to an embodiment, the composition comprises CBG. According to an embodiment, the composition comprises CBC. According to an embodiment, the composition comprises CBN. According to an embodiment, the combinations comprise at least 2 of the terpenes, at least 3, at least 4 or at least 5 of the terpenes.

According to an embodiment, the composition comprises at least one of CBD, THC, CBDa, THCa, CBG, CBC and CBN, CBL, THCV and CBDV and primary terpene is selected from the group consisting of linalool, limonene, eucalyptol, caryophyllene, terpinene and combinations thereof. According to an embodiment, the composition comprises CBD. According to an embodiment, the composition comprises THC. According to an embodiment, the composition comprises CBDa. According to an embodiment, the composition comprises THCa. According to an embodiment, the composition comprises CBG. According to an embodiment, the composition comprises CBC. According to an embodiment, the composition comprises CBN. According to an embodiment, the combinations comprise at least 2 of the terpenes, at least 3, at least 4 or all of the terpenes.

According to an embodiment, the composition comprises at least one cannabinoid selected from the group consisting of CBD, THC, CBDa, THCa, CBG, CBC, CBN, CBL, THCV and CBDV, and at least one primary terpene selected from the group consisting of linalool, limonene, cucalyptol, caryophyllene, terpinene, humulene, terpineol, myrcene, nerolidol and combinations thereof. In some such embodiments, the at least one primary terpene comprises linalool, caryophyellene, limonene, and pinene. In some such embodiments, the at least one primary terpene comprises linalool, caryophyellene, limonene, myrcene, and nerolidol. In some such embodiments, the at least one primary terpene comprises linalool, caryophyellene, limonene, myrcene and terpinene.

According to an embodiment said therapeutic effect treats acute anxiety and/or trauma-related anxiety and said product comprises at least one of CBD, THC, CBC, CBG, CBN, CBL, THCV and CBDV. According to an embodiment said therapeutic effect treats acute anxiety and/or trauma-related anxiety and said product comprises CBD and optionally THC at CBD to THC weight/weight ratio greater than 1, greater than 2, greater than 3, greater than 4 or greater than 5. According to an embodiment said therapeutic effect treats acute anxiety and/or trauma-related anxiety and said product comprises THC and optionally CBD at THC to CBD weight/weight ratio greater than 0.9, greater than 2, greater than 3, greater than 4 or greater than 5. According to various embodiments, said product comprises CBD, comprises THC, comprises CBC, comprises CBG, comprises CBN, comprises CBL, comprises THCV and/or comprises CBDV. According to an embodiment said therapeutic effect treats acute anxiety and/or trauma-related anxiety and the at least one primary is selected from the group consisting of limonene, linalool, terpineol, citronellol, cucalyptol, caryophyllene, geraniol, menthol, pinene, bisabolol, borncol, fenchol, nerolidol and combinations thereof. According to an embodiment, the product comprises at least 2 of these terpenes, at least 3, at least 4, or at least 5 of the terpenes. According to an embodiment, the at least one primary terpene is selected from the group consisting of Limonene, linalool, cucalyptol, humulene and combinations thereof. According to an embodiment, the at least one primary terpene is selected from the group consisting of limonene, linalool, cucalyptol, terpineol, caryophyllene and combinations thereof. According to an embodiment, the at least one primary terpene is selected from the group consisting of limonene, linalool, caryophyllene, bisabolol and combinations thereof. According to various embodiments said at least one primary terpene comprises limonene, linalool, terpineol, citronellol, cucalyptol, caryophyllene, geraniol, menthol, pinene, bisabolol, borneol, fenchol and/or nerolidol.

According to an embodiment, the at least one terpene does not comprise limone or pinene, wherein the composition comprises a secondary terpene selected from the group consisting of humulene, limonene, myrcene and pinene at a concentration of less than 5% by weight of the total amount of terpenes.

According to an embodiment said therapeutic effect treats acute stress and/or post traumatic stress and said product comprises at least one of CBD, THC, CBC, CBG, CBN, CBL, THCV and CBDV. According to an embodiment said therapeutic effect treats acute stress and/or post traumatic stress and said product comprises CBD and optionally THC at CBD to THC weight/weight ratio greater than 1, greater than 2, greater than 3, greater than 4 or greater than 5. According to an embodiment said therapeutic effect treats acute stress and/or post traumatic stress and said product comprises THC and optionally CBD at THC to CBD weight/weight ratio greater than 0.9, greater than 2, greater than 3, greater than 4 or greater than 5. According to various embodiments, said product comprises CBD, comprises THC, comprises CBC, comprises CBG, comprises CBN, comprises CBL, comprises THCV and/or comprises CBDV. According to an embodiment said therapeutic effect treats acute stress and/or post traumatic stress and the at least one primary is selected from the group consisting of linalool, limonene, humulene, terpineol, caryophyllene, bisabolol, borneol, fenchol and combinations thereof. According to an embodiment, the product comprises at least 2 of these terpenes, at least 3, at least 4, or at least 5 of the terpenes. According to an embodiment, the at least one primary terpene is selected from the group consisting of Limonene, linalool, humulene, caryophyllene and combinations thereof. According to an embodiment, the at least one primary terpene is selected from the group consisting of limonene, linalool, humulene, caryophyllene, bisabolol and combinations thereof. According to an embodiment, the primary terpene is linalool. According to various embodiments said at least one primary terpene comprises linalool, limonene, humulene, terpincol, caryophyllene, bisabolol, borncol and/or fenchol.

According to an embodiment, the composition further comprises pinene and/or eucalyptol, wherein said pinene and/or eucalyptol are each provided at a concentration of less than 5% by weight of said total amount of terpenes.

According to an embodiment said therapeutic effect treats acute pain and/or trauma-related pain and said product comprises at least one of CBD, THC, CBC, CBG, CBN, CBL, THCV and CBDV. According to an embodiment said therapeutic effect treats acute pain and/or trauma-related pain and said product comprises CBD and optionally THC at CBD to THC weight/weight ratio greater than 1, greater than 2, greater than 3, greater than 4 or greater than 5. According to an embodiment said therapeutic effect treats acute pain and/or trauma-related pain and said product comprises THC and optionally CBD at THC to CBD weight/weight ratio greater than 0.9, greater than 2, greater than 3, greater than 4 or greater than 5. According to various embodiments, said product comprises CBD, comprises THC, comprises CBC, comprises CBG, comprises CBN, comprises CBL, comprises THCV and/or comprises CBDV. According to an embodiment said therapeutic effect treats acute pain and/or trauma-related pain and the at least one primary is selected from the group consisting of myrcene, linalool, humulene, cucalyptol, caryophyllene, caryophyllene oxide, amyrin, cycloartenol, borneol, sabinene, camphene, nerolidol, terpineol, geraniol, citronellol, camphor, bisabolol, limonene and combinations thereof. According to an embodiment, the product comprises at least 2 of these terpenes, at least 3, at least 4, or at least 5 of the terpenes. According to an embodiment, the at least one primary terpene is selected from the group consisting of myrcene, linalool, caryophyllene and combinations thereof. According to an embodiment, the at least one primary terpene is selected from the group consisting of myrcene, linalool, caryophyllene, eucalyptol, borneol, terpineol and combinations thereof. According to various embodiments said at least one primary terpene comprises myrcene, linalool, humulene, eucalyptol, caryophyllene, caryophyllene oxide, amyrin, cycloartenol, borneol, sabinene, camphene, nerolidol, terpineol, geraniol, citronellol, camphor, bisabolol and/or limonene. According to some such embodiments, the at least one primary terpene is not selected from the group consisting of humulene, bisabolol, borneol, limonene and linalool, wherein the composition comprises a secondary terpene selected from the group consisting of humulene, bisabolol, borncol, limonene and linalool at a concentration of less than 5% by weight of said total amount of terpenes.

According to an embodiment said therapeutic effect comprises neuroprotection and said product comprises at least one of CBD, THC, CBC, CBG, CBN, CBL, THCV and CBDV. According to an embodiment said therapeutic effect comprises neuroprotection and said product comprises CBD and optionally THC at CBD to THC weight/weight ratio greater than 1, greater than 2, greater than 3, greater than 4 or greater than 5. According to an embodiment said therapeutic effect comprises neuroprotection and said product comprises THC and optionally CBD at THC to CBD weight/weight ratio greater than 0.9, greater than 2, greater than 3, greater than 4 or greater than 5. According to various embodiments, said product comprises CBD, comprises THC, comprises CBC, comprises CBG, comprises CBN, comprises CBL, comprises THCV and/or comprises CBDV. According to an embodiment said therapeutic effect comprises neuroprotection and the at least one primary is selected from the group consisting of caryophyllene, pinene, limonene, cucalyptol, myrcene, terpinene, humulene, citronellol, linalool, amyrin, cycloartenol, terpineol and combinations thereof. According to an embodiment, the product comprises at least 2 of these terpenes, at least 3, at least 4, or at least 5 of the terpenes. According to an embodiment, the at least one primary terpene is selected from the group consisting of Caryophyllene, linalool, terpineol, myrcene and combinations thereof. According to an embodiment, the at least one primary terpene is selected from the group consisting of caryophyllene, limonene, cucalyptol, linalool and combinations thereof. According to various embodiments said at least one primary terpene comprises caryophyllene, pinene, limonene, eucalyptol, myrcene, terpinene, humulene, citronellol, linalool, amyrin, cycloartenol and/or terpineol.

According to an embodiment said therapeutic effect comprises antibacterial protection and said product comprises at least one of CBD, THC, CBC, CBG, CBN, CBL, THCV and CBDV. According to an embodiment said therapeutic effect comprises antibacterial protection and said product comprises CBD and optionally THC at CBD to THC weight/weight ratio greater than 1, greater than 2, greater than 3, greater than 4 or greater than 5. According to an embodiment said therapeutic effect comprises antibacterial protection and said product comprises THC and optionally CBD at THC to CBD weight/weight ratio greater than 0.9, greater than 2, greater than 3, greater than 4 or greater than 5. According to various embodiments, said product comprises CBD, comprises THC, comprises CBC, comprises CBG, comprises CBN, comprises CBL, comprises THCV and/or comprises CBDV. According to an embodiment said therapeutic effect comprises antibacterial protection and the at least one primary is selected from the group consisting of terpinolene, pinene, caryophyllene, cucalyptol, camphor, sabinene, humulene, phellandrene, cymene, linalool, amyrin, farnesene, borneol and combinations thereof. According to an embodiment, the product comprises at least 2 of these terpenes, at least 3, at least 4, or at least 5 of the terpenes. According to an embodiment, the at least one primary terpene is selected from the group consisting of Caryophyllene, terpinene, linalool, pinene and combinations thereof. According to an embodiment, the at least one primary terpene is selected from the group consisting of Caryophyllene, terpinene, linalool, Phellandrene, amyrin, farnesene and combinations thereof. According to an embodiment, the at least one primary terpene is selected from the group consisting of terpinolene, pinene, caryophyllene, cucalyptol and combinations thereof. According to various embodiments said at least one primary terpene comprises terpinolene, pinene, caryophyllene, cucalyptol, camphor, sabinene, humulene, phellandrene, cymene, linalool, amyrin, farnesene and/or borncol.

According to an embodiment said therapeutic effect comprises anti-inflammatory protection and said product comprises at least one of CBD, THC, CBC, CBG, CBN, CBL, THCV and CBDV. According to an embodiment said therapeutic effect comprises anti-inflammatory protection and said product comprises CBD and optionally THC at CBD to THC weight/weight ratio greater than 1, greater than 2, greater than 3, greater than 4 or greater than 5. According to an embodiment said therapeutic effect comprises anti-inflammatory protection and said product comprises THC and optionally CBD at THC to CBD weight/weight ratio greater than 0.9, greater than 2, greater than 3, greater than 4 or greater than 5. According to various embodiments, said product comprises CBD, comprises THC, comprises CBC, comprises CBG, comprises CBN, comprises CBL, comprises THCV and/or comprises CBDV. According to an embodiment said therapeutic effect comprises anti-inflammatory protection and the at least one primary is selected from the group consisting of primary terpene is selected from the group consisting of myrcene, limonene, terpinene, pinene, eucalyptol, caryophyllene, guaiol, B-Eudesmol, humulene, citronellol, linalool, amyrin, cycolartenol and combinations thereof. According to an embodiment, the product comprises at least 2 of these terpenes, at least 3, at least 4, or at least 5 of the terpenes. According to an embodiment, the at least one primary terpene is selected from the group consisting of terpinene, pinene, cucalyptol, caryophyllene and combinations thereof. According to an embodiment, the at least one primary terpene is selected from the group consisting of myrcene, caryophylle, humulene, pinene and combinations thereof. According to various embodiments said at least one primary terpene comprises myrcene, limonene, terpinene, pinene, eucalyptol, caryophyllene, guaiol, B-Eudesmol, humulene, citronellol, linalool, amyrin and/or cycolartenol

According to an embodiment said therapeutic effect comprises increased immune response and said product comprises at least one of CBD, THC, CBC, CBG, CBN, CBL, THCV and CBDV. According to an embodiment said therapeutic effect comprises increased immune response and said product comprises CBD and optionally THC at CBD to THC weight/weight ratio greater than 1, greater than 2, greater than 3, greater than 4 or greater than 5. According to an embodiment said therapeutic effect comprises increased immune response and said product comprises THC and optionally CBD at THC to CBD weight/weight ratio greater than 0.9, greater than 2, greater than 3, greater than 4 or greater than 5. According to various embodiments, said product comprises CBD, comprises THC, comprises CBC, comprises CBG, comprises CBN, comprises CBL, comprises THCV and/or comprises CBDV. According to an embodiment said therapeutic effect comprises increased immune response and the at least one primary is selected from the group consisting of terpinolene, linalool, amyrin, pinene, borncol, caryophyllene, geraniol, limonene and combinations thereof. According to an embodiment, the product comprises at least 2 of these terpenes, at least 3, at least 4, or at least 5 of the terpenes. According to an embodiment, the at least one primary terpene is selected from the group consisting of terpinolene, linalool, amyrin, caryophyllene and combinations thereof. According to an embodiment, the at least one primary terpene is selected from the group consisting of pinene, limonene, caryophyllene and combinations thereof. According to various embodiments said at least one primary terpene comprises terpinolene, linalool, amyrin, pinene, borneol, caryophyllene, geraniol and/or limonene.

According to an embodiment said therapeutic effect comprises treatment of conditions and/or symptoms selected from the group consisting of muscle tension, cramps, and pain, and combinations thereof and said product comprises at least one of CBD, THC, CBC, CBG, CBN, CBL, THCV and CBDV. According to an embodiment said therapeutic effect comprises treatment of conditions and/or symptoms selected from the group consisting of muscle tension, cramps, and pain, and combinations thereof and said product comprises CBD and optionally THC at CBD to THC weight/weight ratio greater than 1, greater than 2, greater than 3, greater than 4 or greater than 5. According to an embodiment said therapeutic effect comprises treatment of conditions and/or symptoms selected from the group consisting of muscle tension, cramps, and pain, and combinations thereof and said product comprises THC and optionally CBD at THC to CBD weight/weight ratio greater than 0.9, greater than 2, greater than 3, greater than 4 or greater than 5. According to various embodiments, said product comprises CBD, comprises THC, comprises CBC, comprises CBG, comprises CBN, comprises CBL, comprises THCV and/or comprises CBDV. According to an embodiment said therapeutic effect comprises treatment of conditions and/or symptoms selected from the group consisting of muscle tension, cramps, and pain, and combinations thereof and the at least one primary is selected from the group consisting of myrcene, pinene, nerolidol, sabinene, linalool, humulene, caryophyllene, limonene, bisabolol, citral, cucalyptol, terpineol, geraniol and combinations thereof. According to an embodiment, the product comprises at least 2 of these terpenes, at least 3, at least 4, or at least 5 of the terpenes. According to an embodiment, the at least one primary terpene is selected from the group consisting of myrcene, pinene, limonene and combinations thereof. According to an embodiment, the at least one primary terpene is selected from the group consisting of myrcene, pinene, limonene, nerolidol, linalool, caryophyllene, sabinene and combinations thereof. According to an embodiment, the at least one primary terpene is selected from the group consisting of myrcene, pinene, nerolidol, linalool, humulene and combinations thereof. According to various embodiments said at least one primary terpene comprises myrcene, pinene, nerolidol, sabinene, linalool, humulene, caryophyllene, limonene, bisabolol, citral, eucalyptol, terpineol and/or geraniol.

According to an embodiment, the composition further comprises humulene, limonene, myrcene and/or pinene, wherein said humulene, limonene, myrcene and/or pinene are each provided at a concentration of less than 5% by weight of said total amount of terpenes

According to an embodiment said therapeutic effect comprises treatment of nausea and/or vomiting and said product comprises at least one of CBD, THC, CBC, CBG, CBN, CBL, THCV and CBDV. According to an embodiment said therapeutic effect comprises treatment of nausea and/or vomiting and said product comprises CBD and optionally THC at CBD to THC weight/weight ratio greater than 1, greater than 2, greater than 3, greater than 4 or greater than 5. According to an embodiment said therapeutic effect comprises treatment of nausea and/or vomiting and said product comprises THC and optionally CBD at THC to CBD weight/weight ratio greater than 0.9, greater than 2, greater than 3, greater than 4 or greater than 5. According to various embodiments, said product comprises CBD, comprises THC, comprises CBC, comprises CBG, comprises CBN, comprises CBL, comprises THCV and/or comprises CBDV. According to an embodiment said therapeutic effect comprises treatment of nausea and/or vomiting and the at least one primary is selected from the group consisting of linalool, citronellol, geraniol, menthol, humulene, bisabolol, borneol, fenchol, linalool, limonene, terpineol, pinene, caryophyllene, myrcene, cymene, terpinene and combinations thereof. According to an embodiment, the product comprises at least 2 of these terpenes, at least 3, at least 4, or at least 5 of the terpenes. According to an embodiment, the at least one primary terpene is selected from the group consisting of citronellol, linalool caryophyllene, menthol and combinations thereof. According to an embodiment, the at least one primary terpene is selected from the group consisting of menthol, linalool caryophyllene, limonene, myrcene, nerolidol and combinations thereof. According to various embodiments said at least one primary terpene comprises linalool, citronellol, geraniol, menthol, humulene, bisabolol, borneol, fenchol, linalool, limonene, terpineol, pinene, caryophyllene, myrcene, cymene and/or terpinene.

According to an embodiment, a preparation for preventing, decreasing and/or treating symptoms associated with a stressful event comprises at least one of CBD, THC, CBDa, THCa, CBG, CBC, CBN, CBL, CBDV, THCV wherein said primary terpene is selected from the group consisting of linalool, limonene, eucalyptol, myrcene, caryophyllene, humulene, camphene, borneol, terpineol, pinene, terpinene, citronellol, amyrin, cycloartenol, camphor, sabinene, geraniol, guaiol, β-Eudesmol, friedelin, carvacrol, eugenol, camphor, menthol, iso-menthone, neral, gerial, viridiflorol, germacrene, thymol, Menth-2-en-1-ol, farensol, carotol, myrtenol and combinations thereof. According to an embodiment, the composition forms between 1% by weight and 90% by weight of the preparation. According to an embodiment, the composition forms between 1% and 85% by weight of the preparation, between 1% and 80%, between 1% and 70%, between 1% and 60%, between 1% and 50%, between 1% and 40%, between 1% and 30%, between 1% and 20%, between 1% and 10% by weight of the preparation.

According to an embodiment, the preparation additionally containing an additive selected from the group consisting of antioxidants, emulsifiers and texturizers vegetable oils, plant extracts, honey, sucrose, glucose and fructose, pharmaceutical excipients and combinations thereof.

According to an embodiment, the preparation is selected from the group consisting of tablets, gel capsules, medical patches, cigarettes and vaporizer liquids.

According to an embodiment, the preparation is designed to have an onset time of less than 30 minutes. According to an embodiment, the preparation is designed to have an onset time of less than 25 minutes, less than 20 less than 15, less than 10 minutes, less than 5 minutes.

According to an embodiment, the preparation comprises a first aid kit, according to an embodiment, the preparation comprises an emergency kit. According to an embodiment the preparation is to be part of paramedic tools, part of a surgery room equipment, recovery room equipment, post-operative department equipment, dentist tools, be used by first aid physicians, be provided by psychiatrists.

According to an embodiment, the preparation is designed to have an onset time of more than 60 minutes. According to an embodiment, the preparation is designed to have an onset time of more than 70 minutes, more than 80, more than 90, more than 100, more than 110, more than 120 minutes.

According to an embodiment, the terpene-enriched cannabinoid composition of improved therapeutic effect is a medication for administration to a human subject. According to an embodiment, the terpene-enriched cannabinoid composition of improved therapeutic effect is a veterinary medication.

According to an embodiment, the shelf life of the composition is at least 6 months or at least a year. According to an embodiment, primary terpene degradation in the composition is less than 20% per year.

According to an embodiment, further provided is a product comprising tablets, gel capsules, suppositories, energy drinks, bakery products, medical patches, cigarettes and vaporizer liquids containing the composition.

According to an embodiment, further provided is a commercial product comprising two compositions according to the present invention, which two compositions differ in the content of the primary terpene. According to an embodiment, further provided is a commercial product comprising two compositions according to the present invention, which two compositions comprise different primary terpenes.

According to an embodiment, further provided is a method for treating conditions and/or symptoms associated with a stressful event, comprising administering to a subject in need a therapeutically effective amount of a composition comprising (i) at least one cannabinoid in a specific amount, (ii) primary terpene in a specific amount wherein said at least one primary terpene forms at least 40% by weight of a total amount of terpenes in said composition (iii) at least 5% by weight of a non-cannabinoid, non-terpene carrier and (iv) optionally at least two secondary terpenes. According to an embodiment, the composition provides an enhanced therapeutic effect compared with that of a composition comprising the same cannabinoids amounts and one half, one third, one quarter or one fifth the amount of said the at least one primary terpene. According to one embodment, the enhanced therapeutic effect is compared with that of a composition comprising the same cannabinoids amount and one fifth the amout of the at least one primary terpene.

According to an embodiment, further provided is a composition comprising (i) at least one cannabinoid in a specific amount, (ii) primary terpene in a specific amount wherein said at least one primary terpene forms at least 40% by weight of a total amount of terpenes in said composition (iii) at least 5% by weight of a non-cannabinoid, non-terpene carrier and (iv) optionally at least two secondary terpenes, for use in the treatment of conditions and/or symptoms associated with a stressful event.

According to an embodiment, further provided is the use of a composition comprising (i) at least one cannabinoid in a specific amount, (ii) primary terpene in a specific amount wherein said at least one primary terpene forms at least 40% by weight of a total amount of terpenes in said composition (iii) at least 5% by weight of a non-cannabinoid, non-terpene carrier and (iv) optionally at least two secondary terpenes in the preparation of a medicament for treating conditions and/or symptoms associated with a stressfull event.

According to an embodiment, the composition is for administration in a form selected from the group consisting of cigarettes, vaporizer plant material, vaporizer liquid, extract, tablets, gel capsules, suppositories and combinations thereof. According to an embodiment, the daily dose of the composition comprises about 1 milligram cannabinoid to about 300 milligram, about 2 milligram cannabinoid to about 200 milligram or about 3 milligram cannabinoid to about 100 milligram. According to another embodiment, the daily dose of the composition comprises about 0.5 milligram cannabinoid per kilogram body weight to about 30 milligram cannabinoid per kilogram body weight.

According to an embodiment, the composition comprises less than 5% by weight glycol. According to an embodiment, the composition comprises less than 20% by weight water. According to an embodiment, the composition comprises at least 5% by weight of a non-cannabinoid, non-terpene carrier. According to an embodiment, the non-cannabinoid, non-terpene carrier comprises cellulose and the terpene to cannabinoids weight/weight ratio in the composition is about 0.1 to about 1.0. According to an embodiment, the non-cannabinoid, non-terpene carrier comprises less than 5% by weight cellulose and the terpenes to cannabinoids weight/weight ratio in the composition is about 0.05 to about 1.0,

According to an embodiment, the conditions and/or symptoms are selected from the group consisting of stress, distress, acute anxiety, trauma-related anxiety, depression, acute stress, post trauma stress, acute pain, trauma-related pain, neuronal injury, bacterial infection, muscle tension, inflammation and combination thereof.

According to an embodiment, the therapeutic effect increases immune response.

According to an embodiment, the stressful event includes surgery, dental treatment, unpleasant and/or painful medical procedure, sport injury, battlefield injury, traumatic injury, sudden physical injury, sprain, joint dislocation, bone fracture, childbirth, stressful examination, stressful social event, trauma, major trauma, psychological trauma and combination thereof.

According to an embodiment, the therapeutic effect treats acute or sudden onset conditions and/or symptoms.

According to an embodiment, the therapeutic effect provides a first aid treatment.

According to an embodiment, the therapeutic effect treats delayed conditions and/or symptoms.

According to an embodiment, the composition additionally contains an additive selected from the group consisting of antioxidants, emulsifiers and texturizers vegetable oils, plant extracts, honey, sucrose, glucose and fructose, pharmaceutical excipients and combinations thereof.

According to an embodiment the product comprises tetrahydrocannabinol (THC) and/or tetrahydrocannabinolic acid (THCa), wherein THC and/or THCa is in a total concentration of at least 1% by weight, at least 2%, at least 3%, at least 4%, at least 5%, at least 6%, at least 8%, at least 10%, at least 12%, at least 14%, at least 16%, at least 18%, or at least 20%, at least 30%, at least 40%, at least 50%, at least 60%, at least 70%, at least 80%, or at least 90% by weight.

According to an embodiment, the product comprises cannabidiol (CBD) and/or cannabidiolic acid (CBDa), wherein CBD and/or CBDa is in a total concentration of at least 1% by weight, at least 2%, at least 3%, at least 4%, at least 5%, at least 6%, at least 8%, at least 10%, at least 12%, at least 14%, at least 16%, at least 18% or at least 20%, at least 30%, at least 40%, at least 50%, at least 60%, at least 70%, at least 80%, or at least 90% by weight.

According to an embodiment, the product comprises tetrahydrocannabinol (THC) and/or tetrahydrocannabinolic acid (THCa) and cannabidiol (CBD) and/or cannabidiolic acid (CBDa), wherein, THC and/or THCa is in a total concentration of at least 2.5% by weight, and CBD and/or CBDa is in a total concentration of at least 2.5% by weight; THC and/or THCa in a total concentration of at least 3% by weight, and CBD and/or CBDa in a total concentration of at least 3% by weight; THC and/or THCa in a total concentration of at least 4% by weight, and CBD and/or CBDa in a total concentration of at least 4% by weight; THC and/or THCa in a total concentration of at least 5% by weight, and CBD and/or CBDa in a total concentration of at least 5% by weight; THC and/or THCa in a total concentration of at least 8% by weight, and CBD and/or CBDa in a total concentration of at least 8% by weight; THC and/or THCa in a total concentration of at least 10% by weight, and c CBD and/or CBDa in a total concentration of at least 10% by weight.

According to a related embodiment, said product comprises cannabigerol (CBG) and/or cannabigerol acid (CBGa) in a total concentration of at least 0.1% by weight, at least 0.5%, at least 1%, at least 2%, at least 3%, at least 4%, or at least 5%, at least 6%, at least 8%, at least 10%, at least 12%, at least 14%, at least 16%, at least 18% or at least 20%, at least 30%, at least 40%, at least 50%, at least 60%, at least 70%, at least 80%, or at least 90% by weight. According to a related embodiment, said product comprises cannabinol (CBN) and/or cannabinol acid (CBNa) in a total concentration of at least 0.1% by weight, at least 0.5%, at least 1%, at least 2%, at least 3%, at least 4%, or at least 5%, at least 6%, at least 8%, at least 10%, at least 12%, at least 14%, at least 16%, at least 18% or at least 20%, at least 30%, at least 40%, at least 50%, at least 60%, at least 70%, at least 80%, or at least 90% by weight. According to a related embodiment, said product comprises cannabichromene (CBC) and/or cannabichromenic acid (CBCa) in a total concentration of at least 0.1% by weight, at least 0.5%, at least 1%, at least 2%, at least 3%, at least 4%, or at least 5%, at least 6%, at least 8%, at least 10%, at least 12%, at least 14%, at least 16%, at least 18% or at least 20%, at least 30%, at least 40%, at least 50%, at least 60%, at least 70%, at least 80%, or at least 90% by weight. According to a related embodiment, said product comprises cannabicyclol (CBL) and/or cannabicyclol acid (CBLa) in a total concentration at least 0.1% by weight, at least 0.5%, at least 1%, at least 2%, at least 3%, at least 4%, or at least 5%, at least 6%, at least 8%, at least 10%, at least 12%, at least 14%, at least 16%, at least 18% or at least 20%, at least 30%, at least 40%, at least 50%, at least 60%, at least 70%, at least 80%, or at least 90% by weight. According to a related embodiment, said product comprises tetrahydrocannabivarin (THCV), and/or tetrahydrocannabivarin acid (THCVA) in a total concentration at least 0.1% by weight, at least 0.5%, at least 1%, at least 2%, at least 3%, at least 4%, or at least 5%, at least 6%, at least 8%, at least 10%, at least 12%, at least 14%, at least 16%, at least 18% or at least 20%, at least 30%, at least 40%, at least 50%, at least 60%, at least 70%, at least 80%, or at least 90% by weight. According to a related embodiment, said product comprises cannabidivarin (CBDV) and/or cannabigerovarin acid (CBGVA) in a total concentration at least 0.1% by weight, at least 0.5%, at least 1%, at least 2%, at least 3%, at least 4%, or at least 5%, at least 6%, at least 8%, at least 10%, at least 12%, at least 14%, at least 16%, at least 18% or at least 20%, at least 30%, at least 40%, at least 50%, at least 60%, at least 70%, at least 80%, or at least 90% by weight.

According to an embodiment, the product comprises less than 5% by weight glycol, less than 4%, less than 3%, less than 2%, or less than 1%, by weight glycol. As used herein, the term glycol refers to any glycol, including ethylene glycol, polyethylene glycol, propylene glycol and polypropylene glycol.

According to an embodiment, the product comprises water and water content is less than 30% by weight, less than 25%, less than 20%, less than 15%, less than 12%, less than 10%, or less than 8%. According to an embodiment, water content is at least 1% by weight, at least 2%, at least 3%, at least 4% or at least 5% by weight. According to an embodiment, the product comprises water and water content is more than 50%, more than 60%, more than 70%, more than 80%, more than 90%, or more than 95%.

According to an embodiment, the product comprises chlorophyll. According to an embodiment, the product comprises at least 0.5% chlorophyll, at least 1, % at least 5%, at least 10%, at least 15%, at least 20% chlorophyll. As used herein, the term chlorophyll refers to chlorophyll and degradation products thereof. According to an embodiment, the product comprises at least one flavonoid. According to an embodiment, the product comprises at least two, at least three, at least four, or at least five flavonoids. According to an embodiment, the product comprises at least one of bergamottin, apigenin, amentoflavone, quercetin and piperine. According to an embodiment, the product comprises at least two, at least three, or at least four of bergamottin, apigenin, amentoflavone, quercetin and piperine. According to an embodiment, the product comprises bergamottin. According to an embodiment, the product comprises apigenin. According to an embodiment, the product comprises amentoflavone. According to an embodiment, the product comprises quercetin. According to an embodiment, the product comprises piperine.

According to an embodiment, the composition is liquid. According to an embodiment, the composition is selected from the group consisting of vaporizer cannabis filling liquid, cannabis tablets, cannabis suppositories, cannabis gel capsules, cannabis candies, cannabis drinks and cannabis baked products.

According to an embodiment, the primary terpene of the administered composition is selected from the group consisting of pinene, limonene, linalool, caryophyllene, caryophyllene oxide, myrcene, humulene, borneol, eucalyptol, terpineol, nerolidol, phytol, geraniol, bisabolol, camphene, beta-amyrin, thujone, citronellol, pulegone, cycloartenol, cymene, sabinene, carene, terpinene, fenchol, isopulegol, guaiol, phellandrene, eudesmol, ocimene, cardinene, elemene, gurjunene, farnesene, cardinol, elemene, friedelin, carvacrol, eugenol, camphor, menthol, iso-menthone, neral, gerial, viridiflorol, germacrene, thymol, Menth-2-en-1-ol, farensol, carotol, myrtenol and combinations thereof.

According to an embodiment, the composition comprises at least 5% by weight cellulose and the terpenes to cannabinoids weight/weight ratio in the composition is in the range between from about 0.1 and about 1.0. According to an embodiment, the ratio is greater than 0.1, greater than 0.15, greater than 0.2, greater than 0.25, greater than 0.3, greater than 0.35, greater than 0.4, greater than 0.45, greater than 0.5, greater than 0.55, greater than 0.6, greater than 0.65, greater than 0.7, greater than 0.75, greater than 0.8, greater than 0.85, greater than 0.9, greater than 0.95, greater than 1, greater than 1.2, greater than 1.5, greater than 2.0, greater than 3 or greater than 5. According to an embodiment, the ratio is less than 0.9, less than 0.8, less than 0.7, less than 0.6, less than 0.5, less than 0.4, less than 0.3, less than 0.2 or less than 0.15. According to an embodiment, the composition comprising more than 5% cellulose is selected from the group consisting of cannabis plant material, e.g. cannabis buds or cannabis trim, ground forms thereof, plant material preparation for vaporizers and cannabis cigarette. According to an embodiment, the product comprises a dried cannabis plant material.

According to another embodiment, the non-cannabinoid, non-terpene carrier comprises less than 5% cellulose and terpenes to cannabinoids weight/weight ratio in the composition is in the range between about 0.05 and about 1.0. According to an embodiment, the ratio is greater than 0.1, greater than 0.15, greater than 0.2, greater than 0.25, greater than 0.3, greater than 0.35, greater than 0.4, greater than 0.45, greater than 0.5, greater than 0.55, greater than 0.6, greater than 0.65, greater than 0.7, greater than 0.75, greater than 0.8, greater than 0.85, greater than 0.9, greater than 0.95, greater than 1, greater than 1.2, greater than 1.5, greater than 2.0, greater than 3 or greater than 5. According to an embodiment, the ratio is less than 0.9, less than 0.8, less than 0.7, less than 0.6, less than 0.5, less than 0.4, less than 0.3, less than 0.2, less than 0.15 or less than 0.1. According to an embodiment, the composition comprising less than 5% cellulose is selected from the group consisting of cannabis trichomes, cannabis extracts and products thereof, such as tablets, gel capsules, medical patches, vaporizer liquids and suppositories.

According to an embodiment, the primary terpene and the cannabinoids are present in specific amounts, and the onset time of the therapeutic effect is at least 20% shorter than that of administering a composition comprising the same cannabinoids amounts and one half, one third, one quarter or one fifth the amount of the primary terpene, at least 30%, at least 40%, at least 50% or at least 60% shorter. According to one embodment, the enhanced therapeutic effect is compared with that of a composition comprising the same cannabinoids amount and one fifth the amout of the at least one primary terpene.

According to an embodiment, the primary terpene and the cannabinoids are present in specific amounts, and the magnitude of the therapeutic effect is at least 20% greater compared with that of administering a composition comprising the same cannabinoids amounts and one half, one third, one quarter or one fifth the amount of the primary terpene, at least 30%, at least 40%, at least 50% at least 60%, at least 70%, at least 80%, at least 90% or at least 100% greater. According to one embodment, the enhanced therapeutic effect is compared with that of a composition comprising the same cannabinoids amount and one fifth the amout of the at least one primary terpene.

According to an embodiment, the primary terpene and the cannabinoids are present in specific amounts, and the duration of the therapeutic effect, as measured by methods known in the art, is at least 20% longer compared with that of a composition comprising the same cannabinoids amounts and one half, one third, one quarter or fifth the amount of the primary terpene, at least 30%, at least 40%, at least 50% at least 60%.at least 70%, at least 80%, at least 90% or at least 100% longer. According to an embodiment, the primary terpene, secondary terpenes and the cannabinoids are present in specific amounts, and the duration of the therapeutic effect is at least 20% longer than that of a composition comprising the same cannabinoids amounts, same secondary terpene amounts and one fifth the amount of the primary terpene, at least 30%, at least 40%, at least 50%, at least 60%.at least 70%, at least 80%, at least 90% or at least 100% longer.

According to an embodiment, the primary terpene and the cannabinoids are present in specific amounts, and the frequency of the conditions and/or symptoms is at least 20% smaller compared with that of administering a composition comprising the same cannabinoids amounts and one half, one third, one quarter or one fifth the amount of the primary terpene, at least 30%, at least 40%, at least 50% at least 60%, at least 70%, at least 80%, or at least 90% smaller. According to one embodment, the enhanced therapeutic effect is compared with that of a composition comprising the same cannabinoids amount and one fifth the amout of the at least one primary terpene.

According to an embodiment, the primary terpene and the cannabinoids are present in specific amounts, and the severity of the conditions and/or symptoms is at least 20% smaller compared with that of administering a composition comprising the same cannabinoids amounts and one half, one third, one quarter or one fifth the amount of the primary terpene, at least 30%, at least 40%, at least 50% at least 60%, at least 70%, at least 80%, or at least 90% smaller. According to one embodment, the enhanced therapeutic effect is compared with that of a composition comprising the same cannabinoids amount and one fifth the amout of the at least one primary terpene.

According to an embodiment, the primary terpene and the cannabinoids are present in specific amounts, and the reduced requirement for the composition is a reduction of at least 20% in the dosage required to obtain the same therapeutic effect compared with that of administering a composition comprising the same cannabinoids amounts and one half, one third, one quarter or one fifth the amount of the primary terpene, at least 30%, at least 40%, at least 50% at least 60%, at least 70%, at least 80%, or at least 90% smaller. According to one embodment, the enhanced therapeutic effect is compared with that of a composition comprising the same cannabinoids amount and one fifth the amout of the at least one primary terpene.

According to an embodiment, the primary terpene and the cannabinoids are present in specific amounts, and the consumption of other drugs is reduced by at least 20% compared with that of administering a composition comprising the same cannabinoids amounts and one half, one third, one quarter or one fifth the amount of the primary terpene, at least 30%, at least 40%, at least 50% at least 60%, at least 70%, at least 80%, or at least 90% smaller. According to one embodment, the enhanced therapeutic effect is compared with that of a composition comprising the same cannabinoids amount and one fifth the amout of the at least one primary terpene.

According to an embodiment, the administered composition comprises at least one of CBD, THC, CBDa, THCa, CBG, CBC, CBN CBL, THCV and CBDV, and primary terpene is selected from the group consisting of linalool, limonene, eucalyptol, myrcene, caryophyllene, humulene, camphene, borneol, terpineol, pinene, terpinene, citronellol, amyrin, cycloartenol, camphor, sabinene, geraniol, guaiol, β-Eudesmol, menthol, bisabolol, fenchol, nerolidol, phellandrene, cymene, farnesene, citral and combinations thereof. According to an embodiment, the composition comprises CBD. According to an embodiment, the composition comprises THC. According to an embodiment, the composition comprises CBDa. According to an embodiment, the composition comprises THCa. According to an embodiment, the composition comprises CBG. According to an embodiment, the composition comprises CBC. According to an embodiment, the composition comprises CBN. According to an embodiment, the combinations comprise at least 2 of the terpenes, at least 3, at least 4 or at least 5 of the terpenes.

According to an embodiment, the administered composition comprises at least one of CBD, THC, CBDa, THCa, CBG, CBC, CBN, CBL, THCV and CBDV, and primary terpene is selected from the group consisting of linalool, limonene, eucalyptol, caryophyllene, terpinene and combinations thereof. According to an embodiment, the composition comprises CBD. According to an embodiment, the composition comprises THC. According to an embodiment, the composition comprises CBDa. According to an embodiment, the composition comprises THCa. According to an embodiment, the composition comprises CBG. According to an embodiment, the composition comprises CBC. According to an embodiment, the composition comprises CBN. According to an embodiment, the combinations comprise at least 2 of the terpenes, at least 3, at least 4 or all of the terpenes.

According to an embodiment, the composition comprises at least one cannabinoid selected from the group consisting of CBD, THC, CBDa, THCa, CBG, CBC, CBN, CBL, THCV and CBDV, and at least one primary terpene selected from the group consisting of linalool, limonene, eucalyptol, caryophyllene, terpinene, humulene, terpineol, myrcene, nerolidol and combinations thereof.

In some such embodiments, the at least one primary terpene comprises linalool, caryophyellene, limonene, and pinene.

In some such embodiments, the at least one primary terpene comprises linalool, caryophyellene, limonene, myrcene, and nerolidol.

According to an embodiment said therapeutic effect treats acute anxiety and/or trauma-related anxiety and said product comprises at least one of CBD, THC, CBC, CBG, CBN, CBL, THCV and CBDV. According to an embodiment said therapeutic effect treats acute anxiety and/or trauma-related anxiety and said product comprises CBD and optionally THC at CBD to THC weight/weight ratio greater than 1, greater than 2, greater than 3, greater than 4 or greater than 5. According to an embodiment said therapeutic effect treats acute anxiety and/or trauma-related anxiety and said product comprises THC and optionally CBD at THC to CBD weight/weight ratio greater than 0.9, greater than 2, greater than 3, greater than 4 or greater than 5. According to various embodiments, said product comprises CBD, comprises THC, comprises CBC, comprises CBG, comprises CBN, comprises CBL, comprises THCV and/or comprises CBDV. According to an embodiment said therapeutic effect treats acute anxiety and/or trauma-related anxiety and the at least one primary is selected from the group consisting of limonene, linalool, terpineol, citronellol, eucalyptol, caryophyllene, geraniol, menthol, pinene, bisabolol, borneol, fenchol, nerolidol and combinations thereof. According to an embodiment, the product comprises at least 2 of these terpenes, at least 3, at least 4, or at least 5 of the terpenes. According to an embodiment, the at least one primary terpene is selected from the group consisting of Limonene, linalool, eucalyptol, humulene and combinations thereof. According to an embodiment, the at least one primary terpene is selected from the group consisting of limonene, linalool, eucalyptol, terpineol, caryophyllene and combinations thereof. According to an embodiment, the at least one primary terpene is selected from the group consisting of limonene, linalool, caryophyllene, bisabolol and combinations thereof. According to various embodiments said at least one primary terpene comprises limonene, linalool, terpineol, citronellol, eucalyptol, caryophyllene, geraniol, menthol, pinene, bisabolol, borneol, fenchol and/or nerolidol. According to some such embodiments, the at least one primary terpene does not comprises limonene or pinene and the composition comprises a secondary terpene selected from the group consisting of humulene, limonene, myrcene and pinene at a concentration of less than 5% by weight of the total terpene content.

According to an embodiment said therapeutic effect treats acute stress and/or post traumatic stress and said product comprises at least one of CBD, THC, CBC, CBG, CBN, CBL, THCV and CBDV. According to an embodiment said therapeutic effect treats acute stress and/or post traumatic stress and said product comprises CBD and optionally THC at CBD to THC weight/weight ratio greater than 1, greater than 2, greater than 3, greater than 4 or greater than 5. According to an embodiment said therapeutic effect treats acute stress and/or post traumatic stress and said product comprises THC and optionally CBD at THC to CBD weight/weight ratio greater than 0.9, greater than 2, greater than 3, greater than 4 or greater than 5. According to various embodiments, said product comprises CBD, comprises THC, comprises CBC, comprises CBG, comprises CBN, comprises CBL, comprises THCV and/or comprises CBDV. According to an embodiment said therapeutic effect treats acute stress and/or post traumatic stress and the at least one primary is selected from the group consisting of linalool, limonene, humulene, terpineol, caryophyllene, bisabolol, borneol, fenchol and combinations thereof. According to an embodiment, the product comprises at least 2 of these terpenes, at least 3, at least 4, or at least 5 of the terpenes. According to an embodiment, the at least one primary terpene is selected from the group consisting of Limonene, linalool, humulene, caryophyllene and combinations thereof. According to an embodiment, the at least one primary terpene is selected from the group consisting of limonene, linalool, humulene, caryophyllene, bisabolol and combinations thereof. According to an embodiment, the primary terpene is linalool. According to various embodiments said at least one primary terpene comprises linalool, limonene, humulene, terpineol, caryophyllene, bisabolol, borneol and/or fenchol.

According to an embodiment, the composition further comprises pinene and/or cucalyptol, wherein said pinene and/or eucalyptol are each provided at a concentration of less than 5% by weight of said total amount of terpenes.

According to an embodiment said therapeutic effect treats acute pain and/or trauma-related pain and said product comprises at least one of CBD, THC, CBC, CBG, CBN, CBL, THCV and CBDV. According to an embodiment said therapeutic effect treats acute pain and/or trauma-related pain and said product comprises CBD and optionally THC at CBD to THC weight/weight ratio greater than 1, greater than 2, greater than 3, greater than 4 or greater than 5. According to an embodiment said therapeutic effect treats acute pain and/or trauma-related pain and said product comprises THC and optionally CBD at THC to CBD weight/weight ratio greater than 0.9, greater than 2, greater than 3, greater than 4 or greater than 5. According to various embodiments, said product comprises CBD, comprises THC, comprises CBC, comprises CBG, comprises CBN, comprises CBL, comprises THCV and/or comprises CBDV. According to an embodiment said therapeutic effect treats acute pain and/or trauma-related pain and the at least one primary is selected from the group consisting of myrcene, linalool, humulene, cucalyptol, caryophyllene, caryophyllene oxide, amyrin, cycloartenol, borneol, sabinene, camphene, nerolidol, terpincol, geraniol, citronellol, camphor, bisabolol, limonene and combinations thereof. According to an embodiment, the product comprises at least 2 of these terpenes, at least 3, at least 4, or at least 5 of the terpenes. According to an embodiment, the at least one primary terpene is selected from the group consisting of myrcene, linalool, caryophyllene and combinations thereof. According to an embodiment, the at least one primary terpene is selected from the group consisting of myrcene, linalool, caryophyllene, cucalyptol, borneol, terpineol and combinations thereof. According to various embodiments said at least one primary terpene comprises myrcene, linalool, humulene, cucalyptol, caryophyllene, caryophyllene oxide, amyrin, cycloartenol, borneol, sabinene, camphene, nerolidol, terpineol, geraniol, citronellol, camphor, bisabolol and/or limonene. According to some such embodiments, the primary terpene is not selected from the group consisting of humulene, bisabolol, borncol, limonene and linalool and the composition comprises a secondary terpene selected from the group consisting of humulene, bisabolol, borneol, limonene and linalool at a concentration of S less than 5% by weight of the total terpene content.

According to an embodiment said therapeutic effect comprises neuroprotection and said product comprises at least one of CBD, THC, CBC, CBG, CBN, CBL, THCV and CBDV. According to an embodiment said therapeutic effect comprises neuroprotection and said product comprises CBD and optionally THC at CBD to THC weight/weight ratio greater than 1, greater than 2, greater than 3, greater than 4 or greater than 5. According to an embodiment said therapeutic effect comprises neuroprotection and said product comprises THC and optionally CBD at THC to CBD weight/weight ratio greater than 0.9, greater than 2, greater than 3, greater than 4 or greater than 5. According to various embodiments, said product comprises CBD, comprises THC, comprises CBC, comprises CBG, comprises CBN, comprises CBL, comprises THCV and/or comprises CBDV. According to an embodiment said therapeutic effect comprises neuroprotection and the at least one primary is selected from the group consisting of caryophyllene, pinene, limonene, eucalyptol, myrcene, terpinene, humulene, citronellol, linalool, amyrin, cycloartenol, terpincol and combinations thereof. According to an embodiment, the product comprises at least 2 of these terpenes, at least 3, at least 4, or at least 5 of the terpenes. According to an embodiment, the at least one primary terpene is selected from the group consisting of Caryophyllene, linalool, terpineol, myrcene and combinations thereof. According to an embodiment, the at least one primary terpene is selected from the group consisting of caryophyllene, limonene, eucalyptol, linalool and combinations thereof. According to various embodiments said at least one primary terpene comprises caryophyllene, pinene, limonene, cucalyptol, myrcene, terpinene, humulene, citronellol, linalool, amyrin, cycloartenol and/or terpincol.

According to an embodiment said therapeutic effect comprises antibacterial protection and said product comprises at least one of CBD, THC, CBC, CBG, CBN, CBL, THCV and CBDV. According to an embodiment said therapeutic effect comprises antibacterial protection and said product comprises CBD and optionally THC at CBD to THC weight/weight ratio greater than 1, greater than 2, greater than 3, greater than 4 or greater than 5. According to an embodiment said therapeutic effect comprises antibacterial protection and said product comprises THC and optionally CBD at THC to CBD weight/weight ratio greater than 0.9, greater than 2, greater than 3, greater than 4 or greater than 5. According to various embodiments, said product comprises CBD, comprises THC, comprises CBC, comprises CBG, comprises CBN, comprises CBL, comprises THCV and/or comprises CBDV. According to an embodiment said therapeutic effect comprises antibacterial protection and the at least one primary is selected from the group consisting of terpinolene, pinene, caryophyllene, eucalyptol, camphor, sabinene, humulene, phellandrene, cymene, linalool, amyrin, farnesene, borneol and combinations thereof. According to an embodiment, the product comprises at least 2 of these terpenes, at least 3, at least 4, or at least 5 of the terpenes. According to an embodiment, the at least one primary terpene is selected from the group consisting of Caryophyllene, terpinene, linalool, pinene and combinations thereof. According to an embodiment, the at least one primary terpene is selected from the group consisting of Caryophyllene, terpinene, linalool, Phellandrene, amyrin, farnesene and combinations thereof. According to an embodiment, the at least one primary terpene is selected from the group consisting of terpinolene, pinene, caryophyllene, cucalyptol and combinations thereof. According to various embodiments said at least one primary terpene comprises terpinolene, pinene, caryophyllene, cucalyptol, camphor, sabinene, humulene, phellandrene, cymene, linalool, amyrin, farnesene and/or borneol.

According to an embodiment said therapeutic effect comprises anti-inflammatory protection and said product comprises at least one of CBD, THC, CBC, CBG, CBN, CBL, THCV and CBDV. According to an embodiment said therapeutic effect comprises anti-inflammatory protection and said product comprises CBD and optionally THC at CBD to THC weight/weight ratio greater than 1, greater than 2, greater than 3, greater than 4 or greater than 5. According to an embodiment said therapeutic effect comprises anti-inflammatory protection and said product comprises THC and optionally CBD at THC to CBD weight/weight ratio greater than 0.9, greater than 2, greater than 3, greater than 4 or greater than 5. According to various embodiments, said product comprises CBD, comprises THC, comprises CBC, comprises CBG, comprises CBN, comprises CBL, comprises THCV and/or comprises CBDV. According to an embodiment said therapeutic effect comprises anti-inflammatory protection and the at least one primary is selected from the group consisting of primary terpene is selected from the group consisting of myrcene, limonene, terpinene, pinene, eucalyptol, caryophyllene, guaiol, B-Eudesmol, humulene, citronellol, linalool, amyrin, cycolartenol and combinations thereof. According to an embodiment, the product comprises at least 2 of these terpenes, at least 3, at least 4, or at least 5 of the terpenes. According to an embodiment, the at least one primary terpene is selected from the group consisting of terpinene, pinene, cucalyptol, caryophyllene and combinations thereof. According to an embodiment, the at least one primary terpene is selected from the group consisting of myrcene, caryophylle, humulene, pinene and combinations thereof. According to various embodiments said at least one primary terpene comprises myrcene, limonene, terpinene, pinene, eucalyptol, caryophyllene, guaiol, B-Eudesmol, humulene, citronellol, linalool, amyrin and/or cycolartenol.

According to an embodiment said therapeutic effect comprises increased immune response and said product comprises at least one of CBD, THC, CBC, CBG, CBN, CBL, THCV and CBDV. According to an embodiment said therapeutic effect comprises increased immune response and said product comprises CBD and optionally THC at CBD to THC weight/weight ratio greater than 1, greater than 2, greater than 3, greater than 4 or greater than 5. According to an embodiment said therapeutic effect comprises increased immune response and said product comprises THC and optionally CBD at THC to CBD weight/weight ratio greater than 0.9, greater than 2, greater than 3, greater than 4 or greater than 5. According to various embodiments, said product comprises CBD, comprises THC, comprises CBC, comprises CBG, comprises CBN, comprises CBL, comprises THCV and/or comprises CBDV. According to an embodiment said therapeutic effect comprises increased immune response and the at least one primary is selected from the group consisting of terpinolene, linalool, amyrin, pinene, borneol, caryophyllene, geraniol, limonene and combinations thereof. According to an embodiment, the product comprises at least 2 of these terpenes, at least 3, at least 4, or at least 5 of the terpenes. According to an embodiment, the at least one primary terpene is selected from the group consisting of terpinolene, linalool, amyrin, caryophyllene and combinations thereof. According to an embodiment, the at least one primary terpene is selected from the group consisting of pinene, limonene, caryophyllene and combinations thereof. According to various embodiments said at least one primary terpene comprises terpinolene, linalool, amyrin, pinene, borneol, caryophyllene, geraniol and/or limonene.

According to an embodiment said therapeutic effect comprises treatment of conditions and/or symptoms selected from the group consisting of muscle tension, cramps, and pain, and combinations thereof and said product comprises at least one of CBD, THC, CBC, CBG, CBN, CBL, THCV and CBDV. According to an embodiment said therapeutic effect comprises treatment of conditions and/or symptoms selected from the group consisting of muscle tension, cramps, and pain, and combinations thereof and said product comprises CBD and optionally THC at CBD to THC weight/weight ratio greater than 1, greater than 2, greater than 3, greater than 4 or greater than 5. According to an embodiment said therapeutic effect comprises treatment of conditions and/or symptoms selected from the group consisting of muscle tension, cramps, and pain, and combinations thereof and said product comprises THC and optionally CBD at THC to CBD weight/weight ratio greater than 0.9, greater than 2, greater than 3, greater than 4 or greater than 5. According to various embodiments, said product comprises CBD, comprises THC, comprises CBC, comprises CBG, comprises CBN, comprises CBL, comprises THCV and/or comprises CBDV. According to an embodiment said therapeutic effect comprises treatment of conditions and/or symptoms selected from the group consisting of muscle tension, cramps, and pain, and combinations thereof and the at least one primary is selected from the group consisting of myrcene, pinene, nerolidol, sabinene, linalool, humulene, caryophyllene, limonene, bisabolol, citral, cucalyptol, terpineol, geraniol and combinations thereof. According to an embodiment, the product comprises at least 2 of these terpenes, at least 3, at least 4, or at least 5 of the terpenes. According to an embodiment, the at least one primary terpene is selected from the group consisting of myrcene, pinene, limonene and combinations thereof. According to an embodiment, the at least one primary terpene is selected from the group consisting of myrcene, pinene, limonene, nerolidol, linalool, caryophyllene, sabinene and combinations thereof. According to an embodiment, the at least one primary terpene is selected from the group consisting of myrcene, pinene, nerolidol, linalool, humulene and combinations thereof. According to various embodiments said at least one primary terpene comprises myrcene, pinene, nerolidol, sabinene, linalool, humulene, caryophyllene, limonene, bisabolol, citral, eucalyptol, terpineol and/or geraniol.

According to an embodiment, the composition further comprises humulene, limonene, myrcene and/or pinene, wherein said humulene, limonene, myrcene and/or pinene are each provided at a concentration of less than 5% by weight of said total amount of terpenes.

According to an embodiment said therapeutic effect comprises treatment of nausea and/or vomiting and said product comprises at least one of CBD, THC, CBC, CBG, CBN, CBL, THCV and CBDV. According to an embodiment said therapeutic effect comprises treatment of nausea and/or vomiting and said product comprises CBD and optionally THC at CBD to THC weight/weight ratio greater than 1, greater than 2, greater than 3, greater than 4 or greater than 5. According to an embodiment said therapeutic effect comprises treatment of nausea and/or vomiting and said product comprises THC and optionally CBD at THC to CBD weight/weight ratio greater than 0.9, greater than 2, greater than 3, greater than 4 or greater than 5. According to various embodiments, said product comprises CBD, comprises THC, comprises CBC, comprises CBG, comprises CBN, comprises CBL, comprises THCV and/or comprises CBDV. According to an embodiment said therapeutic effect comprises treatment of nausea and/or vomiting and the at least one primary is selected from the group consisting of linalool, citronellol, geraniol, menthol, humulene, bisabolol, borneol, fenchol, linalool, limonene, terpineol, pinene, caryophyllene, myrcene, cymene, terpinene and combinations thereof. According to an embodiment, the product comprises at least 2 of these terpenes, at least 3, at least 4, or at least 5 of the terpenes. According to an embodiment, the at least one primary terpene is selected from the group consisting of citronellol, linalool caryophyllene, menthol and combinations thereof. According to an embodiment, the at least one primary terpene is selected from the group consisting of menthol, linalool caryophyllene, limonene, myrcene, nerolidol and combinations thereof. According to various embodiments said at least one primary terpene comprises linalool, citronellol, geraniol, menthol, humulene, bisabolol, borneol, fenchol, linalool, limonene, terpineol, pinene, caryophyllene, myrcene, cymene and/or terpinene.

According to an embodiment, the administered composition is designed to have an onset time of less than 30 minutes. According to an embodiment, the administered composition is designed to have an onset time of less than 25 minutes, less than 20 less than 15, less than 10 minutes, less than 5 minutes.

According to an embodiment, the administered composition comprises a first aid kit, according to an embodiment, the preparation comprises an emergency kit. According to an embodiment the preparation is to be part of paramedic tools, part of a surgery room equipment, recovery room equipment, post-operative department equipment, dentist tools, be used by first aid physicians, be provided by psychiatrists.

According to an embodiment, the administered composition is designed to have an onset time of more than 60 minutes. According to an embodiment, the administered composition is designed to have an onset time of more than 70 minutes, more than 80, more than 90, more than 100, more than 110, more than 120 minutes.

According to an embodiment, the method for treating a patient comprises administering to the patient for a first period of time a first terpene-enriched cannabis composition comprising a first cannabinoid at a first cannabinoid amount and a first primary terpene at a first primary terpene amount, followed by administering to said subject for a first period of time a first said composition comprising a first cannabinoid at a first cannabinoid amount and a first primary terpene at a first primary terpene amount, followed by at least one selected from the group consisting of:
administering to said subject for a second period of time a second said composition comprising said first cannabinoid at said first cannabinoid amount or less and a second primary terpene at a second primary terpene amount;
administering to said subject for a second period of time a second said compositon comprising said first cannabinoid at said first cannabinoid amount or less and said first primary terpene at an increased first primary terpene amount;
administering to said subject for a second period of time a second said composition comprising said first cannabinoid at said first cannabinoid amount or less, said first primary terpene at said first primary terpene amount and at least one secondary terpene; and
administering to said subject for a second period of time a second said composition comprising said first cannabinoid at said first cannabinoid amount or less, said first primary terpene at said first primary terpene amount and a second primary terpene.

According to an embodiment, the method further comprises administering to the patient for a third period of time the first terpene-enriched cannabis composition comprising the first cannabinoid amount and a third primary terpene at a third primary terpene amount. According to an embodiment, the third primary terpene is identical to the first primary terpene.

According to another embodiment, the method for treating a patient comprises (i) administering to the patient a first terpene-enriched cannabis composition comprising a first cannabinoid at a first cannabinoid amount and a first primary terpene at a first primary terpene amount and administering to the patient, at least 2 hours later a second terpene-enriched cannabis composition comprising the first cannabinoid amount and a second primary terpenes at a second primary terpene amount. According to another embodiment, the first terpene-enriched cannabis composition is administered for day time and the second terpene-enriched cannabis composition is administered for night time.

According to another embodiment, the method for treating a patient comprises administering to the patient (i) a first terpene-enriched cannabis composition comprising a first cannabinoid at a first cannabinoid amount and a first primary terpene at a first primary terpene amount and (ii) a second terpene-enriched cannabis composition comprising the first cannabinoid and a second primary terpenes at a second primary terpene amount.

According to an embodiment, the method for treating a patient comprises (i) administering to the patient multiple cannabis compositions to find the best working one; (ii) mimicking the best working composition by extracting cannabis plant material to form an extract and blending the extract with suitable terpenes, whereby a mimicking composition is formed and (iii) administering to the patient the mimicking composition. According to an embodiment, the method further comprises removing terpenes from the extract prior to the blending.

According to an embodiment, further provided is a method for treating conditions and/or symptoms associated with a stressful event, comprising administering to a subject in need a therapeutically effective amount of a composition comprising (i) at least one primary terpene in a specific amount; (ii) at least 5% by weight of a non-cannabinoid, non-terpene carrier; (iii) optionally at least two secondary terpenes; and (iv) optionally at least one cannabinoid in a specific amount. According to an embodiment, the composition provides an enhanced therapeutic effect compared with that obtained by administering a composition comprising one half the amount of said the at least one primary terpene. According to an embodiment, the composition comprises less than 20% by weight water.

According to an embodiment, the conditions and/or symptoms are selected from the group consisting of stress, distress, acute anxiety, trauma-related anxiety, depression, acute stress, post traumatic stress, acute pain, trauma-related pain, neuronal injury, bacterial infection, muscle tension, inflammation and combination thereof.

According to an embodiment, the therapeutic effect increases immune response,

According to an embodiment, the stressful event includes a surgery, dental treatment, unpleasant and/or painful medical procedure, sport injury, battlefield injury, traumatic injury, sudden physical injury sprain, joint dislocation, bone fracture, childbirth, stressful examination, stressful social event, trauma, major trauma, psychological trauma and combination thereof.

According to an embodiment, the therapeutic effect treats acute or sudden onset conditions and/or symptoms.

According to an embodiment, the therapeutic effect provides a first aid treatment.

According to an embodiment, the therapeutic effect treats delayed conditions and/or symptoms.

According to an embodiment, the primary terpene is selected from the group consisting of pinene, limonene, linalool, caryophyllene, caryophyllene oxide, myrcene, humulene, borneol, eucalyptol, terpineol, nerolidol, phytol, geraniol, bisabolol, camphene, beta-amyrin, thujone, citronellol, pulegone, cycloartenol, cymene, sabinene, carene, terpinene, fenchol, isopulegol, guaiol, phellandrene, eudesmol, ocimene, cardinene, elemene, gurjunene, farnesene and combinations thereof.

According to an embodiment, the primary terpene is present in specific amounts, and the onset time of the therapeutic effect is at least 20% shorter than that of administering a composition comprising the same cannabinoids amounts and one half the amount of the primary terpene, at least 30%, at least 40%, at least 50% or at least 60% shorter.

According to an embodiment, the primary terpene is present in specific amounts, and the magnitude of the therapeutic effect is at least 20% greater compared with that of administering a composition comprising the same cannabinoids amounts and one half the amount of the primary terpene, at least 30%, at least 40%, at least 50% at least 60%, at least 70%, at least 80%, at least 90% or at least 100% greater. According to an embodiment, the primary terpene is present in specific amounts, and wherein the frequency of the conditions and/or symptoms is at least 20% smaller compared with that of administering a composition comprising the same cannabinoids amounts and one half the amount of the primary terpene, at least 30%, at least 40%, at least 50% at least 60%, at least 70%, at least 80%, or at least 90% smaller. According to an embodiment, the primary terpene is present in specific amounts, and wherein the severity of the conditions and/or symptoms is at least 20% smaller compared with that of administering a composition comprising the same cannabinoids amounts and one half the amount of the primary terpene, at least 30%, at least 40%, at least 50% at least 60%, at least 70%, at least 80%, or at least 90% smaller. According to an embodiment, the primary terpene is present in specific amounts, and wherein the consumption of other drugs is reduced by at least 20% compared with that of administering a composition comprising the same cannabinoids amounts and one half the amount of the primary terpene, at least 30%, at least 40%, at least 50% at least 60%, at least 70%, at least 80%, or at least 90% smaller.

According to an embodiment, the primary terpene is selected from the group consisting of at least two, at least three, at least four or at least five of linalool, limonene, eucalyptol, myrcene, caryophyllene, humulene, camphene, borneol, terpineol, pinene, terpinene, citronellol, amyrin, cycloartenol, camphor, sabinene, geraniol, guaiol, β-Eudesmol.

According to an embodiment, the primary terpene is selected from the group consisting of at least two, at least three, at least four or all least five of linalool, limonene, eucalyptol, caryophyllene, terpinene.

According to an embodiment, the primary terpene is selected from the group consisting of two or three of limonene, linalool, terpineol, citronellol, eucalyptol, caryophyllene, geraniol, menthol, pinene, bisabolol, borneol, fenchol, nerolidol, and the therapeutic effect treats acute anxiety and/or trauma-related anxiety.

According to one embodiment, the at least one primary terpene does not comprise limonene, or pinene, and the composition comprises a secondary terpene selected from the group consisting of humulene, limonene, myrcene and pinene at a concentration of less than 5% by weight of the total terpene content.

According to an embodiment, the at least one primary terpene comprises limonene, linalool, eucalyptol, humulene and combinations thereof. According to an embodiment, the at least one primary terpene comprises limonene, linalool, eucalyptol, terpineol, caryophyllene and combinations thereof. According to an embodiment, the at least one primary terpene comprises limonene, linalool, caryophyllene, bisabolol and combinations thereof. According to an embodiment, the at least one primary terpene comprises linalool.

According to an embodiment, the primary terpene is linalool, and the therapeutic effect treats acute anxiety and/or trauma-related anxiety.

According to an embodiment, the primary terpene is selected from the group consisting of linalool, limonene, humulene, terpineol, caryophyllene, bisabolol, borneol, fenchol and combination thereof and the therapeutic effect treats acute stress and/or post traumatic stress. According to an embodiment, the at least one primary terpene comprises linalool and/or limonene.

According to an embodiment, the composition optionally includes pinene and/or eucalyptol wherein pinene and/or eucalyptol forms less than 5% by weight of the total terpene content.

According to an embodiment, the primary terpene is linalool, and the therapeutic effect treats acute stress and/or post traumatic stress.

According to an embodiment, the primary terpene is selected from the group consisting of at least two, at least three, at least four or at least five of myrcene, linalool, humulene, eucalyptol, caryophyllene, caryophyllene oxide, amyrin, cycloartenol, borneol, sabinene, camphene, nerolidol, terpineol, geraniol, citronellol, camphor, bisabolol, limonene, and the therapeutic effect treats acute pain and/or trauma-related pain. According to an embodiment, the at least one primary terpene comprises myrcene, linalool, caryophyllene and combinations thereof. According to an embodiment, the at least one primary terpene comprises myrcene, linalool, caryophyllene, eucalyptol, borneol, terpineol and combinations thereof. According to one embodiment, the primary terpene is not selected from the group consisting of humulene, bisabolol, borneol, limonene and linalool and the composition comprises a secondary terpene selected from the group consisting of humulene, bisabolol, borneol, limonene and linalool, at a concentration of less than 5% by weight of said total amount of terpenes.

According to an embodiment, the primary terpene is selected from the group consisting of two or three of linalool, eucalyptol, caryophyllene, and the therapeutic effect treats pain and/or acute pain.

According to an embodiment, the primary terpene is selected from the group consisting of at least two, at least three, at least four or at least five of caryophyllene, pinene, limonene, eucalyptol, myrcene, terpinene, humulene, citronellol, linalool, amyrin, cycloartenol, terpineol and the therapeutic effect increases neuroprotection. According to an embodiment, the at least one primary terpene is selected from the group consisting of caryophyllene, linalool, terpineol, myrcene and combination thereof. According to an embodiment, the at least one primary terpene is selected from the group consisting of caryophyllene, limonene, eucalyptol, linalool and combinations thereof.

According to an embodiment, the primary terpene is selected from the group consisting of at least two, at least three, or all four of caryophyllene, limonene, eucalyptol, linalool and the therapeutic effect increases neuroprotection.

According to an embodiment, the primary terpene is selected from the group consisting of at least two, at least three, at least four or at least five of terpinolene, pinene, caryophyllene, eucalyptol, camphor, sabinene, humulene, phellandrene, cymene, linalool, amyrin, farnesene, borneol and the therapeutic effect increases antibacterial protection. According to an embodiment, that at least one primary terpene is selected from the group consisting of caryophyllene, terpinene, linalool, pinene and combination thereof. According to an embodiment, that at least one primary terpene is selected from the group consisting of caryophyllene, terpinene, linalool, Phellandrene, amyrin, farnesene and combinations thereof. According to an embodiment, the at least one primary terpene is selected from the group consisting of terpinolene, pinene, caryophyllene, eucalyptol and combinations thereof.

According to an embodiment, the primary terpene is selected from the group consisting of at least two, at least three, or all four of terpinolene, pinene, caryophyllene, eucalyptol and combinations thereof and the therapeutic effect increases antibacterial protection.

According to an embodiment, the primary terpene is selected from the group consisting of at least two, at least three, at least four or at least five of myrcene, limonene, terpinene, pinene, eucalyptol, caryophyllene, guaiol, B-Eudesmol, humulene, citronellol, linalool, amyrin, cycolartenol and the therapeutic effect increases anti-inflammatory protection. According to an embodiment, the at least one primary terpene is selected from the group consisting of terpinene, pinene, eucalyptol and caryophyllene. According to an embodiment, the at least one primary terpene is selected from the group consisting of myrcene, caryophylle, humulene and pinene.

According to an embodiment, the primary terpene is selected from the group consisting of at least two, at least three, or all four of terpinene, pinene, eucalyptol, caryophyllene and the therapeutic effect increases anti-inflammatory protection.

According to an embodiment, the primary terpene is selected from the group consisting of at least two, at least three, at least four or t least five of terpinolene, linalool, amyrin, pinene, borneol, caryophyllene, geraniol, limonene and the therapeutic effect increase immune response. According to an embodiment, the at least one primary terpene is selected from the group consisting of terpinolene, linalool, amyrin and caryophyllene. According to an embodiment, the at least one primary terpene is selected from the group consisting of pinene, limonene and caryophyllene.

According to an embodiment, the primary terpene is selected from the group consisting of at least two, at least three, or all four of terpinolene, linalool, amyrin, caryophyllene and combinations thereof and the therapeutic effect increase immune response.

According to an embodiment, the primary terpene is selected from the group consisting of at least two, at least three, or all four of myrcene, pinene, nerolidol, sabinene, linalool, humulene, caryophyllene, limonene, bisabolol, citral, eucalyptol, terpineol, geraniol and the therapeutic effect treats muscle tension, cramps, and/or pain.

According to an embodiment, the primary terpene is myrcene and/or pinene and the therapeutic effect treats muscle tension, cramps, and/or pain.

According to an embodiment, the primary terpene is selected from the group consisting of at least two, at least three, or all four of citronellol, geraniol, linalool, limonene and the therapeutic effect treats nausea and/or vomiting.

According to an embodiment, the primary terpene is citronellol and/or linalool and the therapeutic effect treats nausea and/or vomiting.

According to an embodiment, the administrated composition is designed to have an onset time of less than 30 minutes. According to an embodiment, the preparation is designed to have an onset time of less than 25 minutes, less than 20 less than 15, less than 10 minutes, less than 5 minutes.

According to an embodiment, the administrated composition comprises a first aid kit, according to an embodiment, the preparation comprises an emergency kit. According to an embodiment the preparation is to be part of paramedic tools, part of a surgery room equipment, recovery room equipment, post-operative department equipment, dentist tools, be used by first aid physicians, be provided by psychiatrists.

According to an embodiment, the administrated composition is designed to have an onset time of more than 60 minutes. According to an embodiment, the preparation is designed to have an onset time of more than 70 minutes, more than 80, more than 90, more than 100, more than 110, more than 120 minutes.

### Examples 1-20

The Table presents examples of compositions as described herein.

| | | | | Content (% by weight) | | |
|---|---|---|---|---|---|---|
| | | | Primary | | Primary | Therapeutic |
| | Form | Cannabinoid | terpene | Cannabinoid | terpene | effect For: |
| 1 | Cigarette | CBD | Limonene | 3-25 | 1-10 | Acute anxiety |
| 2 | GroundGr | THC | Terpineol | 3-25 | 1-10 | Trauman-related |
| | bud | | | | | pain |
| 3 | Oil [1] | CBG | Linalool | 3-25 | 1-10 | Trauma-related |
| | | | | | | anxiety |
| 4 | Cigarette | THC | Linalool | 3-25 | 1-10 | Acute pain |
| 5 | Oil | THC | Pinene | 3-25 | 1-10 | Muscle tension |
| 6 | Ground | CBD | Myrcene | 3-25 | 1-10 | Muscle |
| | bud | | | | | tension |
| 7 | Cigarette | CBD | Linalool | 3-25 | 1-10 | Preventing post- |
| | | | | | | traumatic stress |
| 8 | Ground | CBD | Pinene | 3-25 | 1-10 | Treating |
| | bud | | | | | inflammation |
| 9 | Cigarette | CBG | Limonene | 3-25 | 1-10 | Acute anxiety |
| 10 | Ground | CBD | Terpinene | 3-25 | 1-10 | Antibacterial |
| | bud | | | | | |
| 11 | Cigarette | CBG | Pinene | 3-25 | 1-10 | Antibacterial |
| 12 | Ground | CBD | Eucalyptol | 3-25 | 1-10 | Acute |
| | bud | | | | | anxiety |
| 13 | Cigarette | CBN | Terpinene | 3-25 | 1-10 | Improving immune |
| | | | | | | response |
| 14 | Oil | CBC | Eucalyptol | 3-25 | 1-10 | Acute pain |
| 15 | Cigarette | CBG | Caryophyllene | 3-25 | 1-10 | Neuroprotection |
| 16 | Ground | CBD | caryophyllene | 3-25 | 1-10 | Trauma-related |
| | bud | | | | | pain |
| 17 | Ground | CBD | caryophyllene | 3-40 | 1-20 | Treating |
| | bud | | | | | inflammation |
| 18 | Ground | THC | Nerolidol | 3-40 | 1-20 | Muscle |
| | bud | | | | | tension |
| 19 | Oil | CBD | Amyrin | 3-40 | 1-20 | Improving immune |
| | | | | | | response |
| 20 | Oil | CBD | Linalool | 3-40 | 1-20 | Acute anxiety |

[1] Oil herein refers to a composition containing an extract of a cannabis plant material.

### Example 21: Treating an Elderly Man Suffering from Acute Anxiety

An elderly man suffering from acute anxiety is treated by administering cannabis cigarettes. The cigarettes comprise CBD and limonene. Administrating involves 2 puffs per dose, twice a day.

### Example 22: Treating an Elderly Man with Acute Pain

An elderly man, suffering from acute pain is treated by administering ground cannabis buds. The buds comprise THC and terpineol.

### Example 23: Treating a Woman with Trauma-Related Anxiety

A woman suffering from trauma-related anxiety is treated by administering cannabis oil sublingually. The cannabis oil contains CBG an linalool. Administrating involves 1 droplet per dose, twice a day.

### Example 24: Treating an Elderly Woman with Trauma-Related Pain

An elderly woman suffering from trauma-related pain is treated by administering cannabis cigarettes. The cigarettes comprise THC and linalool. Administrating involves 2 puffs per dose, twice a day.

### Example 25: Treating a Child with Muscle Tension

A child suffering from muscle tension is treated by administering cannabis oil sublingually. The cannabis oil contains THC and pinene. Administrating involves 1 droplet per dose, twice a day.

### Example 26: Treating a Woman with Muscle Tension

A woman suffering from muscle tension is treated by administering ground cannabis buds in a vaporizer. The ground cannabis contains CBD and myrcene. Administrating involves 3 puffs per dose, 3 times per day.

### Example 27: Treating a Woman at Risk of Post-Traumatic Stress

Post-traumatic stress is prevented in a woman at risk thereof by administering cannabis cigarettes. The cigarettes contain CBD and linalool. Administrating involves 5 puffs per dose, 30 minutes before bedtime.

### Example 28: Treating a Child with Inflammation

A child suffering from inflammation is treated by administering ground cannabis in a vaporizer. The ground cannabis contains CBD and pinene. Administrating involves 3 puffs per dose, 3 times per day.

Thus, the scope of the invention shall include all modifications and variations that may fall within the scope of the attached claims. Other embodiments of the invention will be apparent to those skilled in the art from consideration of the specification and practice of the invention disclosed herein. It is intended that the specification and examples be considered as exemplary only, with a true scope and spirit of the invention being indicated by the following claims.

What is claimed is:

1. A method for preventing an occurrence of a condition or symptom thereof arising due to exposure to a stressful event, comprising administering to a subject in need thereof a therapeutically effective amount of a composition comprising
(i) at least one cannabinoid;
(ii) at least one terpene selected from the group consisting of Pinene, Myrcene, Caryophyllene, Humulene, Nerolidol, Bisabolol, Linalool, Fenchol, Limonene, Terpinene, Terpineol, Eucalyptol, Geraniol, Borneol, Sabinene, Ocimene and combinations thereof, wherein said at least one terpene is present in the composition at a concentration of at least about 20 wt %, based on the weight of the total amount of terpenes in the composition and
(iii) at least 5% by weight of a non-cannabinoid, non-terpene carrier.

2. The method of claim 1, wherein said administering is performed prior to said exposure to said stressful event.

3. The method of claim 1, wherein said administering is performed subsequent to said exposure to said stressful event and prior to said occurrence of said conditions and/or symptoms.

4. The method of claim 1, wherein said composition has an onset time of less than about 30 minutes.

5. The method of claim 1, wherein said composition has an onset time of greater than about 60 minutes.

6. The method of claim 1, wherein a weight/weight ratio of a total amount of terpenes/total amount of cannabinoids in said composition is from 0.05 to 1.

7. The method of claim 1, wherein said composition is liquid at 30° C.

8. The method of claim 1, wherein said at least one cannabinoid is selected from the group consisting of CBD, THC, CBDa, THCa, CBG, CBC, CBN, CBL, THCV, CBDV and combinations thereof.

9. The method of claim 1, wherein said condition or symptom thereof is selected from the group consisting of acute anxiety, trauma-related anxiety, depression, post traumatic stress, acute pain, trauma-related pain, neuronal injury, bacterial infection, muscle tension, inflammation, poor immune response, nausea and/or vomiting and combinations thereof.

10. The method of claim 1, wherein said stressful event is selected from the group consisting of surgery, dental treatment, unpleasant and/or painful medical procedure, sports injury, battlefield injury, traumatic injury, sudden physical injury, sprain, joint dislocation, bone fracture, childbirth, stressful examination, stressful social event, trauma, psychological trauma and combinations thereof.

11. The method of claim 1, wherein said condition or symptom thereof is selected from the group consisting of acute onset conditions and/or symptoms; sudden onset conditions and/or symptoms; and delayed onset conditions and/or symptoms.

12. The method of claim 1, wherein said at least one cannabinoid comprises CBD and optionally THC at a CBD to THC weight/weight ratio of greater than 1:1, wherein said at least one terpene is selected from the group consisting of limonene, linalool, terpineol, eucalyptol, caryophyllene, geraniol, pinene, bisabolol, fenchol, nerolidol and combinations thereof and wherein said condition or symptom thereof is selected from the group consisting of depression, acute anxiety, trauma-related anxiety and combinations thereof.

13. The method of claim 1, wherein said at least one cannabinoid comprises of CBD and optionally THC at a CBD to THC weight/weight ratio of greater than 1:1, wherein said at least one terpene is selected from the group consisting of linalool, limonene, humulene, terpineol, caryophyllene, bisabolol, fenchol and combination thereof and wherein said condition or symptom thereof comprise acute stress and/or post traumatic stress.

14. The method of claim 1, wherein said at least one cannabinoid is selected from the group consisting of CBD, THC, CBC, CBN and CBG, wherein said at least one terpene is selected from the group consisting of myrcene, linalool, humulene, eucalyptol, caryophyllene, nerolidol, terpineol, geraniol, bisabolol, limonene, borneol, sabine, ocimene and combinations thereof and wherein said condition or symptom thereof comprises acute pain and/or trauma-related pain.

15. The method of claim 1, wherein said at least one cannabinoid is selected from the group consisting of CBD, THC, THCa and CBG, wherein said at least one terpene is selected from the group consisting of caryophyllene, pinene, limonene, eucalyptol, myrcene, terpinene, humulene, linalool, terpineol, borneol, sabinene, ocimene and combinations thereof and wherein said condition or symptom thereof comprises neuronal injury.

16. The method of claim 1, wherein said at least one cannabinoid is selected from the group consisting of CBD, THC, CBG, CBC and CBN, wherein said at least one terpene is selected from the group consisting of terpinene, pinene, caryophyllene, eucalyptol, humulene, linalool, limonene, bisabolol and combinations thereof and wherein said condition or symptom thereof comprises bacterial infection.

17. The method of claim 1, wherein at least one cannabinoid is selected from the group consisting of CBD, THC, CBDa, THCa, CBC, CBG and CBN, wherein said at least one terpene is selected from the group consisting of myrcene, limonene, terpinene, pinene, eucalyptol, caryophyllene, humulene, linalool, bisabolol and combinations thereof and wherein said condition or symptom thereof comprises inflammation.

18. The method of claim 1, wherein said at least one cannabinoid is selected from the group consisting of CBD, THC and CBN, wherein said at least one terpene is selected from the group consisting of terpinene, linalool, pinene, caryophyllene, geraniol, limonene, bisabolol and combinations thereof and wherein said condition or symptom thereof comprises poor immune response.

19. The method of claim 1, wherein said at least one cannabinoid is selected from the group consisting of CBD, THC, THCa and CBN, wherein said at least one terepene is selected from the group consisting of myrcene, pinene, nerolidol, linalool, humulene, caryophyllene, limonene, bisabolol, eucalyptol, terpineol, geraniol, borneol, sabinene, ocimene and combinations thereof and wherein said condition or symptom thereof is selected from the group consisting of muscle tension, cramps, pain and combinations thereof.

20. The method of claim 1, wherein said at least one cannabinoid is selected from the group consisting of CBD, THC, and THCa, wherein said at least one terepene is selected from the group consisting of linalool, geraniol, humulene, bisabolol, fenchol, linalool, limonene, terpineol, pinene, caryophyllene, myrcene, terpinene, borneol, sabinene, ocimene and combinations thereof and wherein said condition or symptom thereof comprises nausea and/or vomiting.
